SEQUENCE LISTING (215 sequences)

SEQ ID NO: 1 - protein, 19 aa, Artificial Sequence
  synthetic peptide
CPYCSLQPLALEGSLQKRG

SEQ ID NO: 2 - protein, 19 aa, Artificial Sequence
  synthetic peptide
  PEPTIDE (1)..(1): N-acetylcysteine
XPYCSLQPLALEGSLQKRG

SEQ ID NO: 3 - protein, 19 aa, Artificial Sequence
  synthetic peptide
  PEPTIDE (1)..(1): N-methylcysteine
XPYCSLQPLALEGSLQKRG

SEQ ID NO: 4 - protein, 19 aa, Artificial Sequence
  synthetic peptide
  PEPTIDE (1)..(1): N-ethylcysteine
XPYCSLQPLALEGSLQKRG

SEQ ID NO: 5 - protein, 19 aa, Artificial Sequence
  synthetic peptide
  PEPTIDE (1)..(1): N-propionylcysteine
XPYCSLQPLALEGSLQKRG

SEQ ID NO: 6 - protein, 20 aa, Artificial Sequence
  synthetic peptide
CPYCVQYIKANSKFIGITEL

SEQ ID NO: 7 - protein, 20 aa, Artificial Sequence
  synthetic peptide
  PEPTIDE (1)..(1): N-acetylcysteine
XPYCVQYIKANSKFIGITEL

SEQ ID NO: 8 - protein, 20 aa, Artificial Sequence
  synthetic peptide
  PEPTIDE (1)..(1): N-methylcysteine
XPYCVQYIKANSKFIGITEL

SEQ ID NO: 9 - protein, 20 aa, Artificial Sequence
  synthetic peptide
  PEPTIDE (1)..(1): N-ethylcysteine
XPYCVQYIKANSKFIGITEL

SEQ ID NO: 10 - protein, 20 aa, Artificial Sequence
  synthetic peptide
  PEPTIDE (1)..(1): N-propionylcysteine
XPYCVQYIKANSKFIGITEL

SEQ ID NO: 11 - protein, 19 aa, Artificial Sequence
  synthetic peptide
CHGCSLQPLALEGSLQKRG

SEQ ID NO: 12 - protein, 19 aa, Artificial Sequence
  synthetic peptide
  PEPTIDE (1)..(1): N-acetylcysteine
XHGCSLQPLALEGSLQKRG

SEQ ID NO: 13 - protein, 19 aa, Artificial Sequence
  synthetic peptide
  PEPTIDE (1)..(1): N-methylcysteine
XHGCSLQPLALEGSLQKRG

SEQ ID NO: 14 - protein, 19 aa, Artificial Sequence
  synthetic peptide
  PEPTIDE (1)..(1): N-ethylcysteine
XHGCSLQPLALEGSLQKRG

SEQ ID NO: 15 - protein, 19 aa, Artificial Sequence
  synthetic peptide
  PEPTIDE (1)..(1): N-propionylcysteine
XHGCSLQPLALEGSLQKRG

SEQ ID NO: 16 - protein, 13 aa, Artificial Sequence
  synthetic peptide
CPYCGGFIGLMYY

SEQ ID NO: 17 - protein, 13 aa, Artificial Sequence
  synthetic peptide
  PEPTIDE (1)..(1): N-acetylcysteine
XPYCGGFIGLMYY

SEQ ID NO: 18 - protein, 13 aa, Artificial Sequence
  synthetic peptide
  PEPTIDE (1)..(1): N-methylcysteine
XPYCGGFIGLMYY

SEQ ID NO: 19 - protein, 13 aa, Artificial Sequence
  synthetic peptide
  PEPTIDE (1)..(1): N-ethylcysteine
XPYCGGFIGLMYY

SEQ ID NO: 20 - protein, 13 aa, Artificial Sequence
  synthetic peptide
  PEPTIDE (1)..(1): N-propionylcysteine
XPYCGGFIGLMYY

SEQ ID NO: 21 - protein, 19 aa, Artificial Sequence
  synthetic peptide
CPYCGWYRSPFSRVVHLYR

SEQ ID NO: 22 - protein, 19 aa, Artificial Sequence
  synthetic peptide
  PEPTIDE (1)..(1): N-acetylcysteine
XPYCGWYRSPFSRVVHLYR

SEQ ID NO: 23 - protein, 19 aa, Artificial Sequence
  synthetic peptide
  PEPTIDE (1)..(1): N-methylcysteine
XPYCGWYRSPFSRVVHLYR

SEQ ID NO: 24 - protein, 19 aa, Artificial Sequence
  synthetic peptide
  PEPTIDE (1)..(1): N-ethylcysteine
XPYCGWYRSPFSRVVHLYR

SEQ ID NO: 25 - protein, 19 aa, Artificial Sequence
  synthetic peptide
  PEPTIDE (1)..(1): N-propionylcysteine
XPYCGWYRSPFSRVVHLYR

SEQ ID NO: 26 - protein, 18 aa, Artificial Sequence
  synthetic peptide
CGCSLQPLALEGSLQKRG

SEQ ID NO: 27 - protein, 18 aa, Artificial Sequence
  synthetic peptide
  PEPTIDE (1)..(1): N-acetylcysteine
XGCSLQPLALEGSLQKRG

SEQ ID NO: 28 - protein, 18 aa, Artificial Sequence
  synthetic peptide
  PEPTIDE (1)..(1): N-methylcysteine
XGCSLQPLALEGSLQKRG

SEQ ID NO: 29 - protein, 18 aa, Artificial Sequence
  synthetic peptide
  PEPTIDE (1)..(1): N-ethylcysteine
XGCSLQPLALEGSLQKRG

SEQ ID NO: 30 - protein, 18 aa, Artificial Sequence
  synthetic peptide
  PEPTIDE (1)..(1): N-propionylcysteine
XGCSLQPLALEGSLQKRG

SEQ ID NO: 31 - protein, 4 aa, Artificial Sequence
  synthetic peptide
  misc_feature (2)..(3): Xaa can be any naturally occurring amino acid
  PEPTIDE (4)..(4): C, S or T
CXXX

SEQ ID NO: 32 - protein, 4 aa, Artificial Sequence
  synthetic peptide
  PEPTIDE (1)..(1): C, S or T
  misc_feature (2)..(3): Xaa can be any naturally occurring amino acid
XXXC

SEQ ID NO: 33 - protein, 4 aa, Artificial Sequence
  synthetic peptide
  PEPTIDE (1)..(1): C, N-terminally modified with CH3-CH2-C(=O)
  misc_feature (2)..(3): Xaa can be any naturally occurring amino acid
  PEPTIDE (4)..(4): C, S or T
XXXX

SEQ ID NO: 34 - protein, 5 aa, Artificial Sequence
  synthetic peptide
  PEPTIDE (1)..(1): C, N-terminally modified with CH3-CH2-C(=O)
  misc_feature (2)..(4): Xaa can be any naturally occurring amino acid
  PEPTIDE (5)..(5): C, S or T
XXXXX

SEQ ID NO: 35 - protein, 6 aa, Artificial Sequence
  synthetic peptide
  PEPTIDE (1)..(1): C, N-terminally modified with CH3-CH2-C(=O)
  misc_feature (2)..(5): Xaa can be any naturally occurring amino acid
  PEPTIDE (6)..(6): C, S or T
XXXXXX

SEQ ID NO: 36 - protein, 7 aa, Artificial Sequence
  synthetic peptide
  PEPTIDE (1)..(1): C, N-terminally modified with CH3-CH2-C(=O)
  misc_feature (2)..(6): Xaa can be any naturally occurring amino acid
  PEPTIDE (7)..(7): C, S or T
XXXXXXX

SEQ ID NO: 37 - protein, 8 aa, Artificial Sequence
  synthetic peptide
  PEPTIDE (1)..(1): C, N-terminally modified with CH3-CH2-C(=O)
  misc_feature (2)..(7): Xaa can be any naturally occurring amino acid
  PEPTIDE (8)..(8): C, S or T
XXXXXXXX

SEQ ID NO: 38 - protein, 4 aa, Artificial Sequence
  synthetic peptide
  PEPTIDE (1)..(1): C, N-terminally modified with CH3-C(=O)-
  misc_feature (2)..(3): Xaa can be any naturally occurring amino acid
  PEPTIDE (4)..(4): C, S or T
XXXX

SEQ ID NO: 39 - protein, 5 aa, Artificial Sequence
  synthetic peptide
  PEPTIDE (1)..(1): C, N-terminally modified with CH3-C(=O)-
  misc_feature (2)..(4): Xaa can be any naturally occurring amino acid
  PEPTIDE (5)..(5): C, S or T
XXXXX

SEQ ID NO: 40 - protein, 6 aa, Artificial Sequence
  synthetic peptide
  PEPTIDE (1)..(1): C, N-terminally modified with CH3-C(=O)-
  misc_feature (2)..(5): Xaa can be any naturally occurring amino acid
  PEPTIDE (6)..(6): C, S or T
XXXXXX

SEQ ID NO: 41 - protein, 7 aa, Artificial Sequence
  synthetic peptide
  PEPTIDE (1)..(1): C, N-terminally modified with CH3-C(=O)-
  misc_feature (2)..(6): Xaa can be any naturally occurring amino acid
  PEPTIDE (7)..(7): C, S or T
XXXXXXX

SEQ ID NO: 42 - protein, 8 aa, Artificial Sequence
  synthetic peptide
  PEPTIDE (1)..(1): C, N-terminally modified with CH3-C(=O)-
  misc_feature (2)..(7): Xaa can be any naturally occurring amino acid
  PEPTIDE (8)..(8): C, S or T
XXXXXXXX

SEQ ID NO: 43 - protein, 4 aa, Artificial Sequence
  synthetic peptide
  PEPTIDE (1)..(1): C, N-terminally modified with -CH2-CH3
  misc_feature (2)..(3): Xaa can be any naturally occurring amino acid
  PEPTIDE (4)..(4): C, S or T
XXXX

SEQ ID NO: 44 - protein, 5 aa, Artificial Sequence
  synthetic peptide
  PEPTIDE (1)..(1): C, N-terminally modified with -CH2-CH3
  misc_feature (2)..(4): Xaa can be any naturally occurring amino acid
  PEPTIDE (5)..(5): C, S or T
XXXXX

SEQ ID NO: 45 - protein, 6 aa, Artificial Sequence
  synthetic peptide
  PEPTIDE (1)..(1): C, N-terminally modified with -CH2-CH3
  misc_feature (2)..(5): Xaa can be any naturally occurring amino acid
  PEPTIDE (6)..(6): C, S or T
XXXXXX

SEQ ID NO: 46 - protein, 7 aa, Artificial Sequence
  synthetic peptide
  PEPTIDE (1)..(1): C, N-terminally modified with -CH2-CH3
  misc_feature (2)..(6): Xaa can be any naturally occurring amino acid
  PEPTIDE (7)..(7): C, S or T
XXXXXXX

SEQ ID NO: 47 - protein, 8 aa, Artificial Sequence
  synthetic peptide
  PEPTIDE (1)..(1): C, N-terminally modified with -CH2-CH3
  misc_feature (2)..(7): Xaa can be any naturally occurring amino acid
  PEPTIDE (8)..(8): C, S or T
XXXXXXXX

SEQ ID NO: 48 - protein, 4 aa, Artificial Sequence
  synthetic peptide
  PEPTIDE (1)..(1): C, N-terminally modified with -CH3
  misc_feature (2)..(3): Xaa can be any naturally occurring amino acid
  PEPTIDE (4)..(4): C, S or T
XXXX

SEQ ID NO: 49 - protein, 5 aa, Artificial Sequence
  synthetic peptide
  PEPTIDE (1)..(1): C, N-terminally modified with -CH3
  misc_feature (2)..(4): Xaa can be any naturally occurring amino acid
  PEPTIDE (5)..(5): C, S or T
XXXXX

SEQ ID NO: 50 - protein, 6 aa, Artificial Sequence
  synthetic peptide
  PEPTIDE (1)..(1): C, N-terminally modified with -CH3
  misc_feature (2)..(5): Xaa can be any naturally occurring amino acid
  PEPTIDE (6)..(6): C, S or T
XXXXXX

SEQ ID NO: 51 - protein, 7 aa, Artificial Sequence
  synthetic peptide
  PEPTIDE (1)..(1): C, N-terminally modified with -CH3
  misc_feature (2)..(6): Xaa can be any naturally occurring amino acid
  PEPTIDE (7)..(7): C, S or T
XXXXXXX

SEQ ID NO: 52 - protein, 8 aa, Artificial Sequence
  synthetic peptide
  PEPTIDE (1)..(1): C, N-terminally modified with -CH3
  misc_feature (2)..(7): Xaa can be any naturally occurring amino acid
  PEPTIDE (8)..(8): C, S or T
XXXXXXXX

SEQ ID NO: 53 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with CH3-CH2-C(=O)-O-
CXXX

SEQ ID NO: 54 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with CH3-CH2-C(=O)-O-
CXXXX

SEQ ID NO: 55 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with CH3-CH2-C(=O)-O-
CXXXXX

SEQ ID NO: 56 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with CH3-CH2-C(=O)-O-
CXXXXXX

SEQ ID NO: 57 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with CH3-CH2-C(=O)-O-
CXXXXXXX

SEQ ID NO: 58 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with CH3-C(=O)-O-
CXXX

SEQ ID NO: 59 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with CH3-C(=O)-O-
CXXXX

SEQ ID NO: 60 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with CH3-C(=O)-O-
CXXXXX

SEQ ID NO: 61 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with CH3-C(=O)-O-
CXXXXXX

SEQ ID NO: 62 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with CH3-C(=O)-O-
CXXXXXXX

SEQ ID NO: 63 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with -O-CH2-CH3
CXXX

SEQ ID NO: 64 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with -O-CH2-CH3
CXXXX

SEQ ID NO: 65 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with -O-CH2-CH3
CXXXXX

SEQ ID NO: 66 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with -O-CH2-CH3
CXXXXXX

SEQ ID NO: 67 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with -O-CH2-CH3
CXXXXXXX

SEQ ID NO: 68 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with -O-CH3
CXXX

SEQ ID NO: 69 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with -O-CH3
CXXXX

SEQ ID NO: 70 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with -O-CH3
CXXXXX

SEQ ID NO: 71 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with -O-CH3
CXXXXXX

SEQ ID NO: 72 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with -O-CH3
CXXXXXXX

SEQ ID NO: 73 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with CH3-CH2-C(=O)-NH-
CXXX

SEQ ID NO: 74 - protein, 5 aa, Artificial Sequence
  synthetic peptide
  misc_feature (2)..(3): Xaa can be any naturally occurring amino acid
  PEPTIDE (4)..(4): C, S or T and wherein (5) X is C-terminally modified with CH3-CH2-C(=O)-NH-
  misc_feature (5)..(5): Xaa can be any naturally occurring amino acid
CXXXX

SEQ ID NO: 75 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with CH3-CH2-C(=O)-NH-
CXXXXX

SEQ ID NO: 76 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with CH3-CH2-C(=O)-NH-
CXXXXXX

SEQ ID NO: 77 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with CH3-CH2-C(=O)-NH-
CXXXXXXX

SEQ ID NO: 78 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with CH3-C(=O)-NH-
CXXX

SEQ ID NO: 79 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with CH3-C(=O)-NH-
CXXXX

SEQ ID NO: 80 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with CH3-C(=O)-NH-
CXXXXX

SEQ ID NO: 81 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with CH3-C(=O)-NH-
CXXXXXX

SEQ ID NO: 82 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with CH3-C(=O)-NH-
CXXXXXXX

SEQ ID NO: 83 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with -NH-CH2-CH3
CXXX

SEQ ID NO: 84 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with -NH-CH2-CH3
CXXXX

SEQ ID NO: 85 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with -NH-CH2-CH3
CXXXXX

SEQ ID NO: 86 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with -NH-CH2-CH3
CXXXXXX

SEQ ID NO: 87 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with -NH-CH2-CH3
CXXXXXXX

SEQ ID NO: 88 - protein, 4 aa, Artificial Sequence
  synthetic peptide
  misc_feature (2)..(3): Xaa can be any naturally occurring amino acid
  PEPTIDE (4)..(4): C, S or T and wherein (4) X is C-terminally modified with -NH-CH3
CXXX

SEQ ID NO: 89 - protein, 5 aa, Artificial Sequence
  synthetic peptide
  misc_feature (2)..(4): Xaa can be any naturally occurring amino acid
  PEPTIDE (5)..(5): C, S or T and wherein (5) X is C-terminally modified with -NH-CH3
CXXXX

SEQ ID NO: 90 - protein, 6 aa, Artificial Sequence
  synthetic peptide
  misc_feature (2)..(5): Xaa can be any naturally occurring amino acid
  PEPTIDE (6)..(6): C, S or T and wherein (6) X is C-terminally modified with -NH-CH3
CXXXXX

SEQ ID NO: 91 - protein, 7 aa, Artificial Sequence
  synthetic peptide
  misc_feature (2)..(6): Xaa can be any naturally occurring amino acid
  PEPTIDE (7)..(7): C, S or T and wherein (7) X is C-terminally modified with -NH-CH3
CXXXXXX

SEQ ID NO: 92 - protein, 8 aa, Artificial Sequence
  synthetic peptide
  misc_feature (2)..(7): Xaa can be any naturally occurring amino acid
  PEPTIDE (8)..(8): C, S or T and wherein (8) X is C-terminally modified with -NH-CH3
CXXXXXXX

SEQ ID NO: 93 - protein, 4 aa, Artificial Sequence
  synthetic peptide
  PEPTIDE (1)..(1): C, S or T and wherein (1) X is N-terminally modified with -CH3-CH2-C(=O)
  misc_feature (2)..(3): Xaa can be any naturally occurring amino acid
XXXC

SEQ ID NO: 94 - protein, 5 aa, Artificial Sequence
  synthetic peptide
  PEPTIDE (1)..(1): C, S or T and wherein (1) X is N-terminally modified with -CH3-CH2-C(=O)
  misc_feature (2)..(4): Xaa can be any naturally occurring amino acid
XXXXC

SEQ ID NO: 95 - protein, 6 aa, Artificial Sequence
  synthetic peptide
  PEPTIDE (1)..(1): C, S or T and wherein (1) X is N-terminally modified with -CH3-CH2-C(=O)
  misc_feature (2)..(5): Xaa can be any naturally occurring amino acid
XXXXXC

SEQ ID NO: 96 - protein, 7 aa, Artificial Sequence
  synthetic peptide
  PEPTIDE (1)..(1): C, S or T and wherein (1) X is N-terminally modified with -CH3-CH2-C(=O)
  misc_feature (2)..(6): Xaa can be any naturally occurring amino acid
XXXXXXC

SEQ ID NO: 97 - protein, 8 aa, Artificial Sequence
  synthetic peptide
  PEPTIDE (1)..(1): C, S or T and wherein (1) X is N-terminally modified with -CH3-CH2-C(=O)
  misc_feature (2)..(7): Xaa can be any naturally occurring amino acid
XXXXXXXC

SEQ ID NO: 98 - protein, 4 aa, Artificial Sequence
  synthetic peptide
  PEPTIDE (1)..(1): C, S or T and wherein (1) X is N-terminally modified with CH3-C(=O)-
  misc_feature (2)..(3): Xaa can be any naturally occurring amino acid
XXXC

SEQ ID NO: 99 - protein, 5 aa, Artificial Sequence
  synthetic peptide
  PEPTIDE (1)..(1): C, S or T and wherein (1) X is N-terminally modified with CH3-C(=O)-
  misc_feature (2)..(4): Xaa can be any naturally occurring amino acid
XXXXC

SEQ ID NO: 100 - protein, 6 aa, Artificial Sequence
  synthetic peptide
  PEPTIDE (1)..(1): C, S or T and wherein (1) X is N-terminally modified with CH3-C(=O)-
  misc_feature (2)..(5): Xaa can be any naturally occurring amino acid
XXXXXC

SEQ ID NO: 101 - protein, 7 aa, Artificial Sequence
  synthetic peptide
  PEPTIDE (1)..(1): C, S or T and wherein (1) X is N-terminally modified with CH3-C(=O)-
  misc_feature (2)..(6): Xaa can be any naturally occurring amino acid
XXXXXXC

SEQ ID NO: 102 - protein, 8 aa, Artificial Sequence
  synthetic peptide
  PEPTIDE (1)..(1): C, S or T and wherein (1) X is N-terminally modified with CH3-C(=O)-
  misc_feature (2)..(7): Xaa can be any naturally occurring amino acid
XXXXXXXC

SEQ ID NO: 103 - protein, 4 aa, Artificial Sequence
  synthetic peptide
  PEPTIDE (1)..(1): C, S or T and wherein (1) X is N-terminally modified with -CH2-CH3
  misc_feature (2)..(3): Xaa can be any naturally occurring amino acid
XXXC

SEQ ID NO: 104 - protein, 5 aa, Artificial Sequence
  synthetic peptide
  PEPTIDE (1)..(1): C, S or T and wherein (1) X is N-terminally modified with -CH2-CH3
  misc_feature (2)..(4): Xaa can be any naturally occurring amino acid
XXXXC

SEQ ID NO: 105 - protein, 6 aa, Artificial Sequence
  synthetic peptide
  PEPTIDE (1)..(1): C, S or T and wherein (1) X is N-terminally modified with -CH2-CH3
  misc_feature (2)..(5): Xaa can be any naturally occurring amino acid
XXXXXC

SEQ ID NO: 106 - protein, 7 aa, Artificial Sequence
  synthetic peptide
  PEPTIDE (1)..(1): C, S or T and wherein (1) X is N-terminally modified with -CH2-CH3
  misc_feature (2)..(6): Xaa can be any naturally occurring amino acid
XXXXXXC

SEQ ID NO: 107 - protein, 8 aa, Artificial Sequence
  synthetic peptide
  PEPTIDE (1)..(1): C, S or T and wherein (1) X is N-terminally modified with -CH2-CH3
  misc_feature (2)..(7): Xaa can be any naturally occurring amino acid
XXXXXXXC

SEQ ID NO: 108 - protein, 4 aa, Artificial Sequence
  synthetic peptide
  PEPTIDE (1)..(1): C, S or T and wherein (1) X is N-terminally modified with -CH3
  misc_feature (2)..(3): Xaa can be any naturally occurring amino acid
XXXC

SEQ ID NO: 109 - protein, 5 aa, Artificial Sequence
  synthetic peptide
  PEPTIDE (1)..(1): C, S or T and wherein (1) X is N-terminally modified with -CH3
  misc_feature (2)..(4): Xaa can be any naturally occurring amino acid
XXXXC

SEQ ID NO: 110 - protein, 6 aa, Artificial Sequence
  synthetic peptide
  PEPTIDE (1)..(1): C, S or T and wherein (1) X is N-terminally modified with -CH3
  misc_feature (2)..(5): Xaa can be any naturally occurring amino acid
XXXXXC

SEQ ID NO: 111 - protein, 7 aa, Artificial Sequence
  synthetic peptide
  PEPTIDE (1)..(1): C, S or T and wherein (1) X is N-terminally modified with -CH3
  misc_feature (2)..(6): Xaa can be any naturally occurring amino acid
XXXXXXC

SEQ ID NO: 112 - protein, 8 aa, Artificial Sequence
  synthetic peptide
  PEPTIDE (1)..(1): C, S or T and wherein (1) X is N-terminally modified with -CH3
  misc_feature (2)..(7): Xaa can be any naturally occurring amino acid
XXXXXXXC

SEQ ID NO: 113 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with CH3-CH2-C(=O)-O-
XXXX

SEQ ID NO: 114 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with CH3-CH2-C(=O)-O-
XXXXX

SEQ ID NO: 115 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with CH3-CH2-C(=O)-O-
XXXXXX

SEQ ID NO: 116 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with CH3-CH2-C(=O)-O-
XXXXXXX

SEQ ID NO: 117 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with CH3-CH2-C(=O)-O-
XXXXXXXX

SEQ ID NO: 118 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with CH3-C(=O)-O-
XXXX

SEQ ID NO: 119 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with CH3-C(=O)-O-
XXXXX

SEQ ID NO: 120 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with CH3-C(=O)-O-
XXXXXX

SEQ ID NO: 121 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with CH3-C(=O)-O-
XXXXXXX

SEQ ID NO: 122 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with CH3-C(=O)-O-
XXXXXXXX

SEQ ID NO: 123 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with -O-CH2-CH3
XXXX

SEQ ID NO: 124 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with -O-CH2-CH3
XXXXX

SEQ ID NO: 125 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with -O-CH2-CH3
XXXXXX

SEQ ID NO: 126 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with -O-CH2-CH3
XXXXXXX

SEQ ID NO: 127 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with -O-CH2-CH3
XXXXXXXX

SEQ ID NO: 128 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with -O-CH3
XXXX

SEQ ID NO: 129 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with -O-CH3
XXXXX

SEQ ID NO: 130 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with -O-CH3
XXXXXX

SEQ ID NO: 131 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with -O-CH3
XXXXXXX

SEQ ID NO: 132 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with -O-CH3
XXXXXXXX

SEQ ID NO: 133 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with CH3-CH2-C(=O)-NH-
XXXX

SEQ ID NO: 134 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with CH3-CH2-C(=O)-NH-
XXXXX

SEQ ID NO: 135 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with CH3-CH2-C(=O)-NH-
XXXXXX

SEQ ID NO: 136 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with CH3-CH2-C(=O)-NH-
XXXXXXX

SEQ ID NO: 137 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with CH3-CH2-C(=O)-NH-
XXXXXXXX

SEQ ID NO: 138 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with CH3-C(=O)-NH-
XXXX

SEQ ID NO: 139 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with CH3-C(=O)-NH-
XXXXX

SEQ ID NO: 140 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with CH3-C(=O)-NH-
XXXXXX

SEQ ID NO: 141 - protein, 7 aa, Artificial Sequence
  SEQ ID NO 141
  PEPTIDE (1)..(1): C, S or T
  misc_feature (2)..(6): Xaa can be any naturally occurring amino acid
  PEPTIDE (7)..(7): C, C-terminally modified with CH3-C(=O)-NH-
XXXXXXX

SEQ ID NO: 142 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with CH3-C(=O)-NH-
XXXXXXXX

SEQ ID NO: 143 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with -NH-CH2-CH3
XXXX

SEQ ID NO: 144 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with -NH-CH2-CH3
XXXXX

SEQ ID NO: 145 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with -NH-CH2-CH3
XXXXXX

SEQ ID NO: 146 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with -NH-CH2-CH3
XXXXXXX

SEQ ID NO: 147 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with -NH-CH2-CH3
XXXXXXXX

SEQ ID NO: 148 - protein, 4 aa, Artificial Sequence
  synthetic peptide
  PEPTIDE (1)..(1): C, S or T
  misc_feature (2)..(3): Xaa can be any naturally occurring amino acid
  PEPTIDE (4)..(4): C, C-terminally modified with -NH-CH3
XXXX

SEQ ID NO: 149 - protein, 5 aa, Artificial Sequence
  synthetic peptide
  PEPTIDE (1)..(1): C, S or T
  misc_feature (2)..(4): Xaa can be any naturally occurring amino acid
  PEPTIDE (5)..(5): C, C-terminally modified with -NH-CH3
XXXXX

SEQ ID NO: 150 - protein, 6 aa, Artificial Sequence
  synthetic peptide
  PEPTIDE (1)..(1): C, S or T
  misc_feature (2)..(5): Xaa can be any naturally occurring amino acid
  PEPTIDE (6)..(6): C, C-terminally modified with -NH-CH3
XXXXXX

SEQ ID NO: 151 - protein, 7 aa, Artificial Sequence
  synthetic peptide
  PEPTIDE (1)..(1): C, S or T
  misc_feature (2)..(6): Xaa can be any naturally occurring amino acid
  PEPTIDE (7)..(7): C, C-terminally modified with -NH-CH3
XXXXXXX

SEQ ID NO: 152 - protein, 8 aa, Artificial Sequence
  synthetic peptide
  PEPTIDE (1)..(1): C, S or T
  misc_feature (2)..(7): Xaa can be any naturally occurring amino acid
  PEPTIDE (8)..(8): C, C-terminally modified with -NH-CH3
XXXXXXXX

SEQ ID NO: 153 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 154 - protein, 5 aa, Artificial Sequence
  synthetic peptide
  misc_feature (2)..(4): Xaa can be any naturally occurring amino acid
  PEPTIDE (5)..(5): C, S, or T
CXXXX

SEQ ID NO: 155 - protein, 6 aa, Artificial Sequence
  synthetic peptide
  misc_feature (2)..(5): Xaa can be any naturally occurring amino acid
  PEPTIDE (6)..(6): C, S, or T
CXXXXX

SEQ ID NO: 156 - protein, 7 aa, Artificial Sequence
  synthetic peptide
  misc_feature (2)..(6): Xaa can be any naturally occurring amino acid
  PEPTIDE (7)..(7): C, S, or T
CXXXXXX

SEQ ID NO: 157 - protein, 8 aa, Artificial Sequence
  synthetic peptide
  misc_feature (2)..(7): Xaa can be any naturally occurring amino acid
  PEPTIDE (8)..(8): C, S, or T
CXXXXXXX

SEQ ID NO: 158 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 159 - protein, 5 aa, Artificial Sequence
  synthetic peptide
  PEPTIDE (1)..(1): C, S, or T
  misc_feature (2)..(4): Xaa can be any naturally occurring amino acid
XXXXC

SEQ ID NO: 160 - protein, 6 aa, Artificial Sequence
  synthetic peptide
  PEPTIDE (1)..(1): C, S, or T
  misc_feature (2)..(5): Xaa can be any naturally occurring amino acid
XXXXXC

SEQ ID NO: 161 - protein, 7 aa, Artificial Sequence
  synthetic peptide
  PEPTIDE (1)..(1): C, S, or T
  misc_feature (2)..(6): Xaa can be any naturally occurring amino acid
XXXXXXC

SEQ ID NO: 162 - protein, 8 aa, Artificial Sequence
  synthetic peptide
  PEPTIDE (1)..(1): C, S, or T
  misc_feature (2)..(7): Xaa can be any naturally occurring amino acid
XXXXXXXC

SEQ ID NO: 163 - protein, 4 aa, Artificial Sequence
  synthetic peptide
  PEPTIDE (1)..(4): G, A, V, L, I, M, F, W, P, S, T, C, Y, N, Q, D, E, K, R, and H, or non-natural amino acid
XXXX

SEQ ID NO: 164 - protein, 4 aa, Artificial Sequence
  synthetic peptide
LAVL

SEQ ID NO: 165 - protein, 4 aa, Artificial Sequence
  synthetic peptide
TVQA

SEQ ID NO: 166 - protein, 4 aa, Artificial Sequence
  synthetic peptide
GAVH

SEQ ID NO: 167 - protein, 4 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XAVL

SEQ ID NO: 168 - protein, 4 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
LXVL

SEQ ID NO: 169 - protein, 4 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
LAXL

SEQ ID NO: 170 - protein, 4 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
LAVX

SEQ ID NO: 171 - protein, 4 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XVQA

SEQ ID NO: 172 - protein, 4 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
TXQA

SEQ ID NO: 173 - protein, 4 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
TVXA

SEQ ID NO: 174 - protein, 4 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
TVQX

SEQ ID NO: 175 - protein, 4 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XAVH

SEQ ID NO: 176 - protein, 4 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
GXVH

SEQ ID NO: 177 - protein, 4 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
GAXH

SEQ ID NO: 178 - protein, 4 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
GAVX

SEQ ID NO: 179 - protein, 5 aa, Artificial Sequence
  synthetic peptide
  PEPTIDE (1)..(5): G, A, V, L, I, M, F, W, P, S, T, C, Y, N, Q, D, E, K, R, and H, or non-natural amino acid
XXXXX

SEQ ID NO: 180 - protein, 5 aa, Artificial Sequence
  synthetic peptide
PAFPL

SEQ ID NO: 181 - protein, 5 aa, Artificial Sequence
  synthetic peptide
DQGGE

SEQ ID NO: 182 - protein, 5 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XAFPL

SEQ ID NO: 183 - protein, 5 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
PXFPL

SEQ ID NO: 184 - protein, 5 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
PAXPL

SEQ ID NO: 185 - protein, 5 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
PAFXL

SEQ ID NO: 186 - protein, 5 aa, Artificial Sequence
  synthetic peptide
  PEPTIDE (5)..(5): G, A, V, L, I, M, F, W, P, S, T, C, Y, N, Q, D, E, K, R, and H, or non-natural amino acid
PAFPX

SEQ ID NO: 187 - protein, 5 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XQGGE

SEQ ID NO: 188 - protein, 5 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
DXGGE

SEQ ID NO: 189 - protein, 5 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
DQXGE

SEQ ID NO: 190 - protein, 5 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
DQGXE

SEQ ID NO: 191 - protein, 5 aa, Artificial Sequence
  synthetic peptide
  PEPTIDE (5)..(5): G, A, V, L, I, M, F, W, P, S, T, C, Y, N, Q, D, E, K, R, and H, or non-natural amino acid
DQGGX

SEQ ID NO: 192 - protein, 6 aa, Artificial Sequence
  synthetic peptide
  PEPTIDE (1)..(6): G, A, V, L, I, M, F, W, P, S, T, C, Y, N, Q, D, E, K, R, and H, or non-natural amino acid
XXXXXX

SEQ ID NO: 193 - protein, 6 aa, Artificial Sequence
  synthetic peptide
DIADKY

SEQ ID NO: 194 - protein, 6 aa, Artificial Sequence
  synthetic peptide
  PEPTIDE (1)..(1): G, A, V, L, I, M, F, W, P, S, T, C, Y, N, Q, D, E, K, R, and H, or non-natural amino acid
XIADKY

SEQ ID NO: 195 - protein, 6 aa, Artificial Sequence
  synthetic peptide
  PEPTIDE (2)..(2): G, A, V, L, I, M, F, W, P, S, T, C, Y, N, Q, D, E, K, R, and H, or non-natural amino acid
DXADKY

SEQ ID NO: 196 - protein, 6 aa, Artificial Sequence
  synthetic peptide
  PEPTIDE (3)..(3): G, A, V, L, I, M, F, W, P, S, T, C, Y, N, Q, D, E, K, R, and H, or non-natural amino acid
DIXDKY

SEQ ID NO: 197 - protein, 6 aa, Artificial Sequence
  synthetic peptide
  PEPTIDE (4)..(4): G, A, V, L, I, M, F, W, P, S, T, C, Y, N, Q, D, E, K, R, and H, or non-natural amino acid
DIAXKY

SEQ ID NO: 198 - protein, 6 aa, Artificial Sequence
  synthetic peptide
  PEPTIDE (5)..(5): G, A, V, L, I, M, F, W, P, S, T, C, Y, N, Q, D, E, K, R, and H, or non-natural amino acid
DIADXY

SEQ ID NO: 199 - protein, 6 aa, Artificial Sequence
  synthetic peptide
  PEPTIDE (6)..(6): G, A, V, L, I, M, F, W, P, S, T, C, Y, N, Q, D, E, K, R, and H, or non-natural amino acid
DIADKX

SEQ ID NO: 200 - protein, 7 aa, Artificial Sequence
  synthetic peptide
  PEPTIDE (1)..(1): F,W,Y,H or T
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W,Y,H or T
XXXXXXX

SEQ ID NO: 201 - protein, 7 aa, Artificial Sequence
  synthetic peptide
  PEPTIDE (1)..(1): F,W,Y or H
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W,Y or H
XXXXXXX

SEQ ID NO: 202 - protein, 7 aa, Artificial Sequence
  synthetic peptide
  PEPTIDE (1)..(1): F,W,Y or T
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W,Y or T
XXXXXXX

SEQ ID NO: 203 - protein, 7 aa, Artificial Sequence
  synthetic peptide
  PEPTIDE (1)..(1): F,W or Y
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W or Y
XXXXXXX

SEQ ID NO: 204 - protein, 7 aa, Artificial Sequence
  synthetic peptide
  PEPTIDE (1)..(1): F,W,Y or H
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W,Y or H
XXXXXXX

SEQ ID NO: 205 - protein, 7 aa, Artificial Sequence
  synthetic peptide
  PEPTIDE (1)..(1): F,W,Y,H or T
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W,Y, H or T
XXXXXXX

SEQ ID NO: 206 - protein, 7 aa, Artificial Sequence
  synthetic peptide
  PEPTIDE (1)..(1): F,W or Y
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W or Y
XXXXXXX

SEQ ID NO: 207 - protein, 7 aa, Artificial Sequence
  synthetic peptide
  PEPTIDE (1)..(1): F or W
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W,T or H
XXXXXXX

SEQ ID NO: 208 - protein, 7 aa, Artificial Sequence
  synthetic peptide
  PEPTIDE (1)..(1): F,W,T or H
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F or W
XXXXXXX

SEQ ID NO: 209 - protein, 4 aa, Artificial Sequence
  synthetic peptide
  PEPTIDE (1)..(1): C, or N-terminally modified C
XPYC

SEQ ID NO: 210 - protein, 4 aa, Artificial Sequence
  synthetic peptide
  PEPTIDE (1)..(1): C, or N-terminally modified C
XHGC

SEQ ID NO: 211 - protein, 19 aa, Artificial Sequence
  synthetic peptide
CHGCGWYRSPFSRVVHLYR

SEQ ID NO: 212 - protein, 20 aa, Artificial Sequence
  synthetic peptide
  PEPTIDE (1)..(1): Xaa is N-acetylcysteine
XCHGCGWYRSPFSRVVHLYR

SEQ ID NO: 213 - protein, 19 aa, Artificial Sequence
  synthetic peptide
GWYRSPFSRVVHLYRCPYC

SEQ ID NO: 214 - protein, 19 aa, Artificial Sequence
  synthetic peptide
  PEPTIDE (19)..(19): Xaa is NH-methylcysteine
GWYRSPFSRVVHLYRCPYX

SEQ ID NO: 215 - protein, 19 aa, Artificial Sequence
  synthetic peptide
  PEPTIDE (19)..(19): Xaa is NH-ethylcysteine
GWYRSPFSRVVHLYRCPYX

(19) United States
(12) Patent Application Publication
(10) Pub. No.: US 2023/0113747 A1
(43) Pub. Date: Apr. 13, 2023

(54) IMMUNOGENIC PEPTIDES WITH AN OXIDOREDUCTASE MOTIF COMPRISING A MODIFIED CYSTEINE

(71) Applicant: IMCYSE SA, Angleur (Liege) (BE)

(72) Inventor: Milos ERAK, Brussels (BE)

(21) Appl. No.: 17/611,290

(22) PCT Filed: May 18, 2020
(86) PCT No.: PCT/EP2020/063860

§ 371 Date: Nov. 15, 2021

(30) Foreign Application Priority Data
May 16, 2019 (EP) 19174917.5

(51) Int. Cl. C07K 14/33; A61P 37/02; A61P 37/06; C12N 5/0783

(52) U.S. Cl. CPC C07K 14/33; A61P 37/02; A61P 37/06; C12N 5/0638; A61K 2039/627

(57) ABSTRACT

The invention relates to immunogenic peptides comprising T-cell epitopes and oxidoreductase motifs comprising a modified cysteine, and their use in regulating the immune response in subjects.

### CROSS-REFERENCE TO RELATED APPLICATIONS

This application is the U.S. National Phase of International Application No. PCT/EP2020/063860, filed May 18, 2020, which claims the benefit of European Patent Application No. 19174917.5, filed May 16, 2019, each of which is incorporated herein by reference in its entirety.

### REFERENCE TO SEQUENCE LISTING SUBMITTED ELECTRONICALLY:

The content of the electronically submitted sequence listing (Name: 2752_0136 Sequence_Listing.txt; Size: 79.1 kilobytes; and Date of Creation: Nov. 14, 2021) filed with the application is incorporated herein by reference in its entirety.

### BACKGROUND OF THE INVENTION

Several strategies have been described to prevent the generation of an unwanted immune response against an antigen. WO2008017517 describes a new strategy using peptides comprising an MHC class II antigen of a given antigenic protein and a typical C-XX-[CST] or [CST]-XX-C oxidoreductase peptide motif. These peptides convert CD4+ T cells into a cell type with cytolytic properties called cytolytic CD4+ T cells. These cells are capable to kill via triggering apoptosis the antigen presenting cells (APC), which present the antigen from which the peptide is derived. WO2008/017517 demonstrates this concept for allergies and auto-immune diseases such as type I diabetes, where insulin can act as an auto-antigen. WO2009101207 and Carlier et al. (2012) Plos one 7,10 e45366 further describe the antigen specific cytolytic cells in more detail, as well as the mode of action of those peptides, which act by reducing disulfide bridges at the surface of CD4 T cells, in an antigen-specific manner.

In addition to the peptides comprising an MHC class II epitope of e.g. an allergen or antigen, WO2012069568 further discloses the possibility of using NKT cell epitopes linked to an oxidoreductase motif, binding the CD1d receptor and resulting in activation of cytolytic antigen-specific NKT cells, which have been shown to eliminate, in an antigen-specific manner, APC presenting said specific antigen.

WO2016059236 and WO2017182528 further disclose modified peptides wherein an additional histidine or a tryptophan is present in the proximity of the oxidoreductase motif, thereby increasing the stability of the oxidoreductase motif.

WO2008017517 also discloses that the oxidoreductase motif may comprise amino adds with modified side chains, such as methylated cysteine, which is converted into cysteine with free thiol groups in vivo.

Prior art however remains silent on putative modifications of cysteine on other groups than the SH side chain. N- or C-terminal modification of cysteines in the oxidoreductase motif has not been reported.

### SUMMARY OF THE INVENTION

The present invention provides immunogenic peptides comprising a T-cell epitope of an antigen and an oxidoreductase motif with a N-or C-terminal modified cysteine.

The present invention relates to the following aspects:

Aspect 1. An immunogenic peptide, said immunogenic peptide comprising:
a) an oxidoreductase peptide motif of formula (I) or (II),
b) a T-cell epitope of an antigenic protein, and
c) a linker between a) and b) of between 0 and 7 amino acids;

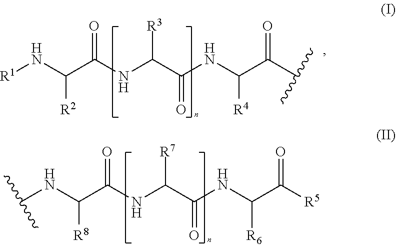

wherein the wavy line ( [symbol] ) in formula (I) indicates the point of attachment to the amino group of the N-terminal end of the linker (c) or the epitope (b), and wherein the wavy line ( [symbol] ) in formula II indicates the point of attachment to the carbonyl group of the C-terminal end of the linker (c) or the epitope (b);
wherein:
R¹ is selected from the group comprising CH₃—CH₂—C(═O)—, CH₃—C(═O)—, —CH₂—CH₃, and —CH₃, preferably CH₃—CH₂—C(═O)—, CH₃—C(═O)—, or —CH₃;
R² and R⁴ are each independently selected from the group comprising —CH₂—SH, —CH₂—OH, and —CH(OH)—CH₃; wherein at least one of R² or R⁴ is —CH₂—SH;
R³ and R⁷ are each independently selected from the group comprising H, —CH₃, —(CH₂)₃—NH—C(═NH)—NH₂, —CH₂—C(═O)—NH₂, —CH₂—C(═O)—OH, —(CH₂)₂—C(═O)—NH₂, —CH₂—SH, —(CH₂)₂—C(═O)—OH, —CH₂—(1H-imidazol-4-yl), —CH₂—CH(CH₃)₂, —(CH₂)₄—NH₂, —CH(CH₃)—CH₂—CH₃, —CH₂—OH, —CH(CH₃)₂, —CH(OH)—CH₃, —CH₂-phenyl, —CH₂-1H-indol-3-yl, —(CH₂)₂—S—CH₃, and —CH₂-(4-hydroxyphenyl), or wherein NH—R³ or NH—R⁷ together with the carbon atom to which they are attached form a pyrrolidinyl ring;
R⁵ is selected from the group comprising CH₃—CH₂—C(═O)—O—, CH₃—C(═O)—O—, —O—CH₂—CH₃, —O—CH₃, CH₃—CH₂—C(═O)—NH—, CH₃—C(═O)—NH—, —NH—CH₂—CH₃, and —NH—CH₃;
R⁶ and R⁸ are each independently selected from the group comprising —CH₂—SH, —CH₂—OH, and —CH(OH)—CH₃; wherein at least one of R⁶ or R⁸ is —CH₂—SH,
   wherein n and m are each independently an integer selected from the group comprising: 1, 2, 3, 4, 5 and 6.

In all embodiments disclosed herein, said oxidoreductase peptide motif of formula (I) or (II)

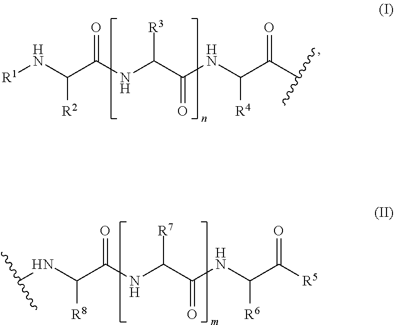

can also be summarized and represented as

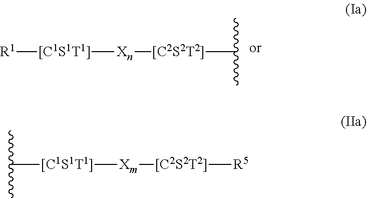

wherein the wavy line ( [symbol] ) in formula (Ia) indicates the point of attachment to the amino group of the N-terminal end of the linker (c) or the epitope (b), and wherein the wavy line ( [symbol] ) in formula IIa indicates the point of attachment to the carbonyl group of the C-terminal end of the linker (c) or the epitope (b);
wherein:
R¹ is selected from the group comprising CH₃—CH₂—C(═O)—, CH₃—C(═O)—, —CH₂—CH₃, and —CH₃, preferably CH₃—CH₂—C(═O)—, CH₃—C(═O)—, or —CH₃;
R⁵ is selected from the group comprising CH₃—CH₂—C(═O)—O—, CH₃—C(═O)—O—, —O—CH₂—CH₃, —O—CH₃, CH₃—CH₂—C(═O)—NH—, CH₃—C(═O)—NH—, —NH—CH₂—CH₃, and —NH—CH₃;
R¹—[C¹S¹T¹] represent an amino acid moiety respectively selected from cysteine, serine or threonine, that has been chemically modified through R¹ via N-acetylation, N-methylation, N-ethylation or N-propionylation, preferably wherein said amino acid is a cysteine chemically modified through N-acetylation, N-methylation, N-ethylation or N-propionylation;
[C²S²T²]—R⁵ represent an amino acid moiety respectively selected from cysteine, serine or threonine, that has been chemically modified through C-terminal substitution R⁵ by acetyl, methyl, ethyl or propionyl groups of it's C-terminal amide or acid groups, preferably wherein said amino acid is a cysteine chemically modified through C-terminal substitution by acetyl, methyl, ethyl or propionyl groups of it's C-terminal amide or acid groups;
wherein in each formula Ia or IIa at least one of the [C¹S¹T¹] or [C²S²T²] amino acid moieties is a cysteine, more preferably wherein both amino acid moieties are a cysteine as in: R¹—C¹—Xₙ—C²— (Formula Ib) or —C¹—Xₘ—C²—R⁵ (Formula IIb);
X corresponds to any amino acid moiety,
wherein n and m are each independently an integer selected from the group comprising: 1, 2, 3, 4, 5 and 6.

Aspect 2. The immunogenic peptide according to aspect 1, wherein R² is —CH₂—SH, and R⁶ is —CH₂—SH, i.e. wherein in [C¹S¹T¹] or [C²S²T²], a cysteine is selected instead of a serine or threonine.

Aspect 3. The immunogenic peptide according to any one of aspects 1 or 2, wherein R³ and R⁷ are each independently selected from: —CH₂—(1H-imidazol-4-yl), —(CH₂)₃—NH—C(═NH)—NH₂, and —(CH₂)₄—NH₂.

Aspect 4. The immunogenic peptide according to any one of aspects 1 to 3, wherein R³ and R⁷ are each independently selected from —CH₂-(4-hydroxphenyl), NH—R³ together with the carbon atom to which there are attached form a pyrrolidinyl ring, or NH—R⁷ together with the carbon atom to which they are attached form a pyrrolidinyl ring.

Aspect 5. The immunogenic peptide according to any one of aspects 1 to 4, wherein n or m is 2.

Aspect 6. The immunogenic peptide according to any one of aspects 1 to 5, wherein R⁴ or R⁸ are —CH₂—SH.

Aspect 7. The immunogenic peptide according to any one of aspects 1 to 6, wherein the oxidoreductase motif is of formula (I).

Aspect 8. The immunogenic peptide according to any one of aspects 1 to 7, wherein the T-cell epitope does not naturally comprise a cysteine, serine, or threonine residue within its sequence and/or within a region of 11 amino acids N-terminally or C-terminally of the T-cell epitope.

Aspect 9. The immunogenic peptide according to any one of aspects 1 to 8, wherein said oxidoreductase motif does not naturally occur within a region of 11 amino acids N-terminally or C-terminally of the T-cell epitope in said antigenic protein.

Aspect 10. The immunogenic peptide according to any one of aspects 1 to 9, wherein the T-cell epitope does not naturally comprise said oxidoreductase motif.

Aspect 11. The immunogenic peptide according to any one of aspects 1 to 10, wherein said T cell epitope of an antigenic protein is an MHC class II T cell epitope or an NKT cell epitope.

Aspect 12. The immunogenic peptide according to any one of aspects 1 to 11, wherein said epitope fits into the binding cleft of the MHC class II molecule or the CD1d molecule.

Aspect 13. The immunogenic peptide according to any one of aspects 1 to 12, wherein said epitope has a length of between 7 and 30 amino acids, preferably between 7 and 25 amino acids, more preferably between 7 and 20 amino acids.

Aspect 14. The immunogenic peptide according to any one of aspects 1 to 13, having a length of between 10 and 75 amino acids, preferably between 10 and 50 amino acids, more preferably between 10 and 40 amino acids, more preferably between 10 and 30 amino acids, and even more preferably between 10 and 25 amino acids.

Aspect 15. The immunogenic peptide according to any one of aspects 1 to 14, wherein the linker is of between 0 and 4 amino acids.

Aspect 16. The immunogenic peptide according to any one of aspects 1 to 15, wherein said antigenic protein is an auto-antigen, a soluble allofactor, an alloantigen shed by a graft, an antigen of an intracellular pathogen, an antigen of a viral vector used for gene therapy or gene vaccination, a tumor-associated antigen or an allergen.

Aspect 17. The immunogenic peptide according to any one of aspects 1 to 16, for use in medicine.

Aspect 18. The immunogenic peptide according to any one of aspects 1 to 17, for use in treating and/or prevention of an autoimmune disease, of an infection with an intracellular pathogen, of a tumor, of an allograft rejection, or of an immune response to a soluble allofactor, to an allergen exposure or to a viral vector used for gene therapy or gene vaccination.

Aspect 19. A method for preparing an immunogenic peptide according to any one of aspects 1 to 18, comprising the steps of:
a1) synthesizing said immunogenic peptide, e.g. by conventional peptide synthesis for example using a conventional peptide synthesizer;
   or
a2) providing a peptide consisting of a T-cell epitope of an antigenic protein, and
b2) linking at the N- or C- terminal end of said peptide a compound of formula (III) or (IV) respectively, wherein R¹ to R⁷, m and n are as defined in claim 1 such that said compound of formula (III) or (IV) and said epitope are either adjacent to each other or separated by a linker of at most 7 amino acids;

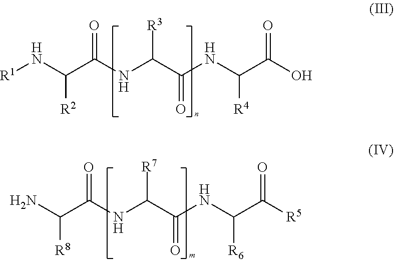

or
a3) providing a peptide consisting of a T-cell epitope of an antigenic protein, and
b3) linking at the N- or C-terminal end of said peptide with a compound of formula (V) or (VI) respectively, wherein R¹⁰ is hydrogen or R¹¹ is a NH₂ or OH and R² to R⁴ and R⁶ to R⁸, m and n are as defined in claim 1, such that said motif and said compound of formula (V) or (VI) are either adjacent to each other or separated by a linker of at most 7 amino acids, and replacing said R¹⁰ or R¹¹ of said compound of formula (V) or (VI) with at least one CH₃—CH₂—C(═O)—, CH₃—C(═O)—, —CH₂—CH₃, or —CH₃ group,

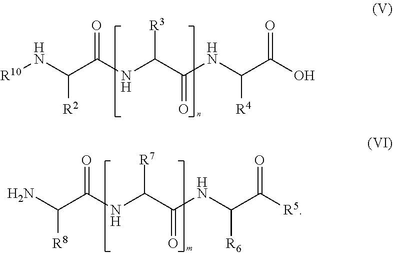

Aspect 20. A method for preparing an immunogenic peptide according to any one of aspects 1 to 18, comprising the steps of: synthesizing said immunogenic peptide starting from natural amino acids and a chemically modified cysteine selected from the group consisting of: N-acetylated cysteine, N-methylated cysteine, N-ethylated cysteine, N-propionylated cysteine, or a cysteine in which its C-terminally C-terminal amide or acid groups have been substituted by acetyl, methyl, ethyl or propionyl groups.

Aspect 21. A method for preparing an immunogenic peptide according to any one of aspects 1 to 18, comprising the steps of:
a2) providing a peptide consisting of a T-cell epitope (b) of an antigenic protein, optionally coupled to a linker (c) of between 0 and 7 amino acids.
b2) providing an oxidoreductase motif having the following general structure: C¹—Xₙ—C²— or —C¹—Xₘ—C₂,
   wherein X corresponds to any amino acid moiety;
wherein n and m both are 2;
wherein the C-terminal hyphen (-) in formula (Ib) indicates the point of attachment to the amino group of the N-terminal end of the linker (c) or the epitope (b), and wherein the N-terminal hyphen (-) in formula IIb indicates the point of attachment to the carbonyl group of the C-terminal end of the linker (c) or the epitope (b); and b3) chemically modifying said C¹ amino acid residue through N-acetylation, N-methylation, N-ethylation or N-propionylation, or
chemically modifying said C² amino acid residue through C-terminal substitution by acetyl, methyl, ethyl or propionyl groups of it's C-terminal amide or acid groups.

Aspect 22. A method for obtaining a population of antigen-specific cytolytic CD4+ T cells, against APC presenting said antigen, the method comprising the steps of:
providing peripheral blood cells;
contacting said cells with an immunogenic peptide according to any one of aspects 1 to 18
expanding said cells in the presence of IL-2.

Aspect 23. A method for obtaining a population of antigen-specific NKT cells, the method comprising the steps of:
providing peripheral blood cells;
contacting said cells with an immunogenic peptide according to any one of aspects 1 to 18
expanding said cells in the presence of IL-2.

Aspect 24. A method for obtaining a population of antigen-specific cytolytic CD4+ T cells, against APC presenting said antigen, the method comprising the steps of:
providing an immunogenic peptide according to any one of aspects 1 to 18
administering said peptide to a subject; and
obtaining said population of antigen-specific cytolytic CD4+ T cells from said subject.

Aspect 25. A method for obtaining a population of antigen-specific NKT cells, the method comprising the steps of:
providing an immunogenic peptide according to any one of aspects 1 to 18
administering said peptide to a subject; and
obtaining said population of antigen-specific NKT cells from said subject.

Aspect 26. The population of antigen-specific cytolytic CD4+T cells or NKT cells obtainable by the method of any one of aspects 22 to 25 for use in the treatment and/or prevention of an autoimmune disease, an infection with an intracellular pathogen, a tumor, an allograft rejection, or an immune response to a soluble allofactors, to an allergen exposure or to a viral vector used for gene therapy or gene vaccination.

Aspect 27. A method of treating and/or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of administering the immunogenic peptide according to any one of aspects 1 to 18 or the cell population according to aspect 26 to said individual.

Aspect 28. A method of treating or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of:
providing peripheral blood cells of said individual,
contacting said cells with an antigenic peptide according to any of aspects 1 to 18
expanding said cells, and
administering said expanded cells to said individual.

The peptides of the present invention have the advantage that activity of the modified oxidoreductase peptide motifs disclosed herein have an enhanced oxidoreductase activity as compared to known oxidoreductase peptide motifs of the [CST]-X_{n/m}—C or C—X_{n/m}-[CST] type. The peptides of the present invention have hence a greater potency and a greater capacity to generate cytolytic CD4+ T cells as compared to the prior art peptides.

### BRIEF DESCRIPTION OF THE FIGURES

The present invention is illustrated by the following figures which are to be considered for illustrative purpose only and in no way limit the invention to the embodiments disclosed therein.

FIG. 1: represents the comparisons of the oxidoreductase activities of Peptide 1 having the sequence CPYCSLQPLALEGSLQKRG and Peptide 2 having the sequence N-Acetyl-CPYCSLQPLALEGSLQKRG. DTT is used as a positive control.

FIG. 2: represents comparisons of the oxidoreductase activities of Peptide 6 having the sequence CPYCVQYIKANSKFIGITEL and Peptide 7 having the sequence N-Acetyl-CPYCVQYIKANSKFIGITEL. DTT is used as a positive control.

FIG. 3: represents comparisons of the oxidoreductase activities of peptides 21 to 25 (see table 5 for detailed sequences). DTT is used as a positive control.

FIG. 4: represents comparisons of the oxidoreductase activities of peptides 26 and 27 (see table 6 for detailed sequences). DTT is used as a positive control.

FIG. 5: represents comparisons of the oxidoreductase activities of peptides 28 to 30 (see table 7 for detailed sequences). DTT is used as a positive control.

### DETAILED DESCRIPTION OF THE INVENTION

The present invention will be described with respect to particular embodiments but the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would have to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e.

anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 10 and 200 amino acids, connected by peptide bonds, but which can comprise non-amino acid structures.

The term “immunogenic peptide” as used herein refers to a peptide that is immunogenic, i.e. that comprises a T-cell epitope capable of eliciting an immune response.

Peptides according to the invention can contain any of the conventional 20 amino acids or modified versions thereof, or can contain non-naturally occurring amino-acids incorporated by chemical peptide synthesis or by chemical or enzymatic modification.

The terms “oxidoreductase motif”, “oxidoreductase peptide motif”, “thiol-oxidoreductase motif”, “thioreductase motif”, “thiooxidoreductase motif” or “redox motif” are used herein as synonymous terms and refer to motifs involved in the transfer of electrons from one molecule (the reductant, also called the hydrogen or electron donor) to another (the oxidant, also called the hydrogen or electron acceptor). Typical oxidoreductase peptide motifs are depicted as C-X_{n/m}-[CST] or [CST]-X_{n/m}—C peptide motifs in which C stands for cysteine, S for serine, T for threonine and X for any amino acid moiety or residue, and wherein n is an integer selected from the group comprising: 1, 2, 3, 4, 5 or 6, typically 1, 2 or 3. In the present invention, one of the C or [CST] residues has been modified so as to carry a acetyl, methyl, ethyl or propionyl group, either on the N-terminal amide of the amino acid residue of the motif or on the C-terminal carboxy group.

This results in oxidoreductase motifs according to the following general formulas:

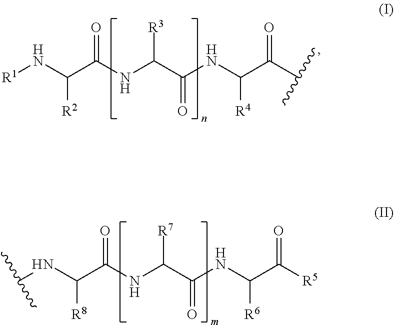

wherein the wavy line ( [symbol] ) in formula (I) indicates the point of attachment to the amino group of the N-terminal end of the linker (c) or the epitope (b), and wherein the wavy line ( [symbol] ) in formula II indicates the point of attachment to the carbonyl group of the C-terminal end of the linker (c) or the epitope (b);

wherein:
R¹ is selected from the group comprising CH₃—CH₂—C(═O)— (propionyl), CH₃—C(═O)— (acetyl), —CH₂—CH₃ (ethyl), and —CH₃ (methyl); preferably CH₃—CH₂—C(═O)— (propionyl), CH₃—C(═O)— (acetyl) and —CH₃ (methyl);
R² and R⁴ are each independently selected from the group comprising —CH₂—SH (thereby forming a cysteine residue), —CH₂—OH (thereby forming a serine residue), and —CH(OH)—CH₃ (thereby forming a threonine residue); wherein at least one of R² or R⁴ is —CH₂—SH (thereby forming a cysteine residue);
R³ and R⁷ are each independently selected from the group comprising H (thereby forming a glycine residue), —CH₃ (thereby forming an alanine residue), —(CH₂)₃—NH—C(═NH)—NH₂ (thereby forming an arginine residue), —CH₂—C(═O)—NH₂ (thereby forming an asparagine residue), —CH₂—C(═O)—OH (thereby forming an aspartic acid residue), —(CH₂)₂—C(═O)—NH₂ (thereby forming a glutamine residue), —CH₂—SH (thereby forming a cysteine residue), —(CH₂)₂—C(═O)—OH (thereby forming a glutamic acid residue), —CH₂—(1H-imidazol-4-yl) (thereby forming a histidine residue), —CH₂—CH(CH₃)₂ (thereby forming a leucine residue), —(CH₂)₄—NH₂ (thereby forming a lysine residue), —CH(CH₃)—CH₂—CH₃ (thereby forming an isoleucine residue), —CH₂—OH (thereby forming a serine residue), —CH(CH₃)₂ (thereby forming a valine residue), —CH(OH)—CH₃ (thereby forming a threonine residue), —CH₂-phenyl (thereby forming a phenylalanine residue), —CH₂—1H-indol-3-yl (thereby forming a tryptophan residue), —(CH₂)₂—S—CH₃ (thereby forming a methionine residue), and —CH₂—(4-hydroxyphenyl) (thereby forming a tyrosine residue), or wherein NH—R³ or NH—R⁷ together with the carbon atom to which they are attached form a pyrrolidinyl ring (thereby forming a proline residue);
R⁵ is selected from the group comprising CH₃—CH₂—C(═O)—O— (acid group substituted by propionyl), CH₃—C(═O)—O— (acid group substituted by acetyl), —O—CH₂—CH₃ (acid group substituted by ethyl), —O—CH₃ (acid group substituted by methyl), CH₃—CH₂—C(═O)—NH—(amide group substituted by propionyl), CH₃—C(═O)—NH— (amide group substituted by acetyl), —NH—CH₂—CH₃ (amide group substituted by ethyl), and —NH—CH₃ (amide group substituted by methyl);
R⁶ and R⁸ are each independently selected from the group comprising —CH₂—SH (thereby forming a cysteine residue), —CH₂—OH (thereby forming a serine residue), and —CH(OH)—CH₃ (thereby forming a threonine residue); wherein at least one of R⁶ or R⁸ is —CH₂—SH (thereby forming a cysteine residue),
wherein n and m are each independently an integer selected from the group comprising: 1, 2, 3, 4, 5 and 6.

In all embodiments disclosed herein, said oxidoreductase peptide motif of formula (I) or (II) depicted above can also be summarized and represented as:

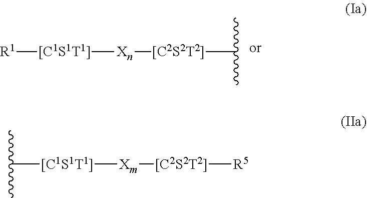

wherein
wherein the wavy line ( [symbol] ) in formula (Ia) indicates the point of attachment to the amino group of the N-terminal end of the linker (c) or the epitope (b), and wherein the wavy line ( [symbol] ) in formula IIa indicates the point of attachment to the carbonyl group of the C-terminal end of the linker (c) or the epitope (b);
R¹ is selected from the group comprising CH₃—CH₂—C(═O)—, CH₃—C(═O)—, —CH₂—CH₃, and —CH₃;
R⁵ is selected from the group comprising CH₃—CH₂—C(═O)—O—, CH₃—C(═O)—O—, —O—CH₂—CH₃, —O—CH₃, CH₃—CH₂—C(═O)—NH—, CH₃—C(═O)—NH—, —NH—CH₂—CH₃, and —NH—CH₃;
[C¹S¹T¹] represent an amino acid moiety selected from cysteine, serine or threonine;
[C²S²T²] represent an amino acid moiety selected from cysteine, serine or threonine;
wherein in each formula Ia or IIa at least one of the [C¹S¹T1] or [C²S²T²] amino acid moieties is a cysteine;
X corresponds to any amino acid moiety,
wherein n and m are each independently an integer selected from the group comprising: 1, 2, 3, 4, 5 and 6.

Cysteines in the above recited oxidoreductase motifs hence represents either cysteine but can also represents another amino acid with a thiol group such as mercaptovaline, homocysteine or other natural or non-natural amino acids with a thiol function. In order to have reducing activity, the one or more cysteines present in the oxidoreductase motif should not occur as part of a cystine disulfide bridge.

Preferably said X in formula Ia or IIa above is selected from the group consisting of: G, A, V, L, I, M, F, W, P, S, T, C, Y, N, Q, D, E, K, and R, H, or non-natural amino acid.

More preferably at least one X in formula Ia or IIa above is a basic amino acid selected from: K, R, H or a non-natural basic amino acid. The term “basic amino acid” refers to any amino acid that acts like a Bronsted-Lowry and Lewis base, and includes the natural basic amino acids Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)0H(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

In a preferred embodiment of formula Ia or IIa, integer n or m is 1 and X is any amino acid selected from the group consisting of: G, A, V, L, I, M, F, W, P, S, T, C, Y, N, Q, D, E, K, R, and H, or non-natural amino acids. Preferably, X in motif is any amino acid except for C, S, or T. In a specific embodiment, X in the motif is a basic amino acid selected from: H, K, or R, or a non-natural basic amino acid as defined therein.

In a preferred embodiment of formula Ia or IIa, integer n or m is 2, thereby creating an internal X¹X² amino acid couple within the oxidoreductase motif. X¹ and X², each individually, can be any amino acid selected from the group consisting of: G, A, V, L, I, M, F, W, P, S, T, C, Y, N, Q, D, E, K, R, and H, or non-natural amino acids. Preferably,

X¹ and X² in said motif is any amino acid except for C, S, or T. In a specific embodiment, at least one of X¹ or X² in said motif is a basic amino acid selected from: H, K, or R, or a non-natural basic amino acid as defined herein. In another specific embodiment, at least one of X¹ or X² in said motif is P or Y. Specific examples of the internal X¹X² amino acid couple within the oxidoreductase motif: PY, HY, KY, RY, PH,

PK, PR, HG, KG, RG, HH, HK, HR, GP, HP, KP, RP, GH, GK, GR, GH, KH, and RH.

In a preferred embodiment of formula Ia or IIa, integer n or m is 3, thereby creating an internal X¹X²X³ amino acid stretch within the oxidoreductase motif. X¹, X², and X³, each individually can be any amino acid selected from the group consisting of: G, A, V, L, I, M, F, W, P, S, T, C, Y, N, Q, D, E, K, R, and H, or non-natural amino acids.

Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein.

Specific examples of the internal X¹X²X³ amino acid stretch within the oxidoreductase motif are: XPY, PXY, and PYX, wherein X can be can be any amino acid, such as in:
KPY, RPY, HPY, GPY, APY, VPY, LPY, IPY, MPY, FPY, WPY, PPY, SPY, TPY, CPY, YPY, NPY, QPY, DPY, EPY, and KPY; or
PKY, PRY, PHY, PGY, PAY, PVY, PLY, PIY, PMY, PFY, PWY, PPY, PSY, PTY, PCY, PYY, PNY, PQY, PDY, PEY, and PLY; or
PYK, PYR, PYH, PYG, PYA, PYV, PYL, PYI, PYM, PYF, PYW, PYP, PYS, PYT, PYC, PYY, PYN, PYQ, PYD, PYE, and PYL;

XHG, HXG, and HGX, wherein X can be can be any amino acid, such as in:
KHG, RHG, HHG, GHG, AHG, VHG, LHG, IHG, MHG, FHG, WHG, PHG, SHG, THG, CHG, YHG, NHG, QHG, DHG, EHG, and KHG; or
HKG, HRG, HHG, HGG, HAG, HVG, HLG, HIG, HMG, HFG, HWG, HPG, HSG, HTG, HCG, HYG, HNG, HQG, HDG, HEG, and HLG; or
HGK, HGR, HGH, HGG, HGA, HGV, HGL, HGI, HGM, HGF, HGW, HGP, HGS, HGT, HGC, HGY, HGN, HGQ, HGD, HGE, and HGL;

XGP, GXP, and GPX, wherein X can be can be any amino acid, such as in:
KGP, RGP, HGP, GGP, AGP, VGP, LGP, IGP, MGP, FGP, WGP, PGP, SGP, TGP, CGP, YGP, NGP, QGP, DGP, EGP, and KGP; or
GKP, GRP, GHP, GGP, GAP, GVP, GLP, GIP, GMP, GFP, GWP, GPP, GSP, GTP, GCP, GYP, GNP, GQP, GDP, GEP, and GLP; or
GPK, GPR, GPH, GPG, GPA, GPV, GPL, GPI, GPM, GPF, GPW, GPP, GPS, GPT, GPC, GPY, GPN, GPQ, GPD, GPE, and GPL;

XGH, GXH, and GHX, wherein X can be can be any amino acid, such as in:
KGH, RGH, HGH, GGH, AGH, VGH, LGH, IGH, MGH, FGH, WGH, PGH, SGH, TGH, CGH, YGH, NGH, QGH, DGH, EGH, and KGH; or
GKH, GRH, GHH, GGH, GAH, GVH, GLH, GIH, GMH, GFH, GWH, GPH, GSH, GTH, GCH, GYH, GNH, GQH, GDH, GEH, and GLH; or
GHK, GHR, GHH, GHG, GHA, GHV, GHL, GHI, GHM, GHF, GHW, GHP, GHS, GHT, GHC,

GHY, GHN, GHQ, GHD, GHE, and GHL;

XGF, GXF, and GFX, wherein X can be can be any amino acid, such as in:
KGF, RGF, HGF, GGF, AGF, VGF, LGF, IGF, MGF, FGF, WGF, PGF, SGF, TGF, CGF, YGF, NGF, QGF, DGF, EGF, and KGF; or
GKF, GRF, GHF, GGF, GAF, GVF, GLF, GIF, GMF, GFF, GWF, GPF, GSF, GTF, GCF, GYF, GNF, GQF, GDF, GEF, and GLF; or
GFK, GFR, GFH, GFG, GFA, GFV, GFL, GFI, GFM, GFF, GFW, GFP, GFS, GFT, GFC, GFY, GFN, GFQ, GFD, GFE, and GFL;

XRL, RXL, and RLX, wherein X can be can be any amino acid, such as in:
KRL, RRL, HRL, GRL, ARL, VRL, LRL, IRL, MRL, FRL, WRL, PRL, SRL, TRL, CRL, YRL, NRL, QRLRL, DRL, ERL, and KRL; or
GKF, GRF, GHF, GGF, GAF, GVF, GLF, GIF, GMF, GFF, GWF, GPF, GSF, GTF, GCF, GYF, GNF, GQF, GDF, GEF, and GLF; or
RLK, RLR, RLH, RLG, RLA, RLV, RLL, RLI, RLM, RLF, RLW, RLP, RLS, RLT, RLC, RLY, RLN, RLQ, RLD, RLE, and RLL;

XHP, HXP, and HPX, wherein X can be can be any amino acid, such as in:
KHP, RHP, HHP, GHP, AHP, VHP, LHP, IHP, MHP, FHP, WHP, PHP, SHP, THP, CHP, YHP, NHP, QHP, DHP, EHP, and KHP; or
HKP, HRP, HHP, HGP, HAF, HVF, HLF, HIF, HMF, HFF, HWF, HPF, HSF, HTF, HCF, HYP, HNF, HQF, HDF, HEF, and HLP; or
HPK, HPR, HPH, HPG, HPA, HPV, HPL, HPI, HPM, HPF, HPW, HPP, HPS, HPT, HPC, HPY, HPN, HPQ, HPD, HPE, and HPL;

In a preferred embodiment of formula Ia or IIa, integer n or m is 4, thereby creating an internal X¹X²X³X⁴ amino acid stretch within the oxidoreductase motif. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In a specific embodiment, at least one of X¹, X², X³ or X⁴ in said motif is a basic amino acid selected from: H, K, or R, or a non-natural basic amino acid as defined herein.

Specific examples are: LAVL, TVQA or GAVH and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴; wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

In a preferred embodiment of formula Ia or Ha, integer n or m is 5, thereby creating an internal X¹X²X³X⁴X⁵ amino acid stretch within the oxidoreductase motif. X¹, X², X³, X⁴ and X⁵ each individually can be any amino acid selected from the group consisting of:
G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid selected from: H, K, or R, or a non-natural basic amino acid as defined herein.

Specific examples are: PAFPL or DQGGE and their variants such as: X¹AFPL, PX²FPL, PAX³PL, PAFX⁴L, or PAFPX⁵; X¹QGGE, DX²GGE, DQX³GE, DQGX⁴E, or DQGGX⁵, wherein X′, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein.

In a preferred embodiment of formula Ia or IIa, integer n or m is 6, thereby creating an internal X¹X²X³X⁴X⁵X⁶ amino acid stretch within the oxidoreductase motif X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T. In a specific embodiment, at least one of X¹, X², X³ X⁴, X⁵ or X⁶ in said motif is a basic amino acid selected from: H, K, or R, or a non-natural basic amino acid as defined herein.

Specific examples are: DIADKY or variants thereof such as: X¹IADKY, DX²ADKY, DIX³DKY, DIAX⁴KY, DIADX⁵Y, or DIADKX⁶, wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein.

In the context of the present invention or as disclosed herein, amino acid moieties, preferably cysteines, refer to L-amino acids, D-amino acids or racemic mixtures of L- or D-amino acids.

The term N-acetylcysteine refers to the IV-acetyl derivative of the amino acid cysteine. As used herein, “N-acetylcysteine” (NAC) or “acetylcysteine”, includes any form of acetylcysteine, including N-acetyl-L-cysteine (CAS No. 616-91-1), N-acetyl-D-cysteine (CAS No. 26117-28-2), and racemic N-acetylcysteine or a (racemic) mixture of N-acetyl-L-cysteine and N-acetyl-D-cysteine).

The term N-methylcysteine refers to the N-methyl derivative of the amino acid cysteine. As used herein, “N-methylcysteine” or “methylcysteine”, includes any forms of N-methyl-L-cysteine (CAS No. 4026-48-6), N-methyl-D-cysteine, and racemic N-methylcysteine or a (racemic) mixture of N-methyl-L-cysteine and N-methyl-D-cysteine). S-methylcysteine is explicitly excluded in all the embodiments of the present invention since it may result in possible disruption of the thioredox activity,

The term N-ethylcysteine refers to the N-ethyl derivative of the amino acid cysteine. As used herein, “N-ethylcysteine” or “ethylcysteine”, includes any form of ethylcysteine, including N-ethyl-L-cysteine, N-ethyl-D-cysteine, and racemic N-ethylcysteine or a (racemic) mixture of N-ethyl-L-cysteine and N-ethyl-D-cysteine).

The term N-propionylcysteine refers to the N-propionyl derivative of the amino acid cysteine. As used herein, “N-propionylcysteine” or “propionylcysteine”, includes any form of propionylcysteine, including N-propionyl-L-cysteine (CAS No. 2885-79-2), N-propionyl-D-cysteine, and racemic N-propionylcysteine or a (racemic) mixture of N-propionyl-L-cysteine and N-propionyl-D-cysteine). The term “antigen” as used herein refers to a structure of a macromolecule, typically a protein (with or without polysaccharides) or made of proteic composition comprising one or more hapten(s) and comprising T or NKT cell epitopes.

The term “antigenic protein” as used herein refers to a protein comprising one or more T or NKT cell epitopes. The antigenic protein according to the invention can be an auto-antigen, a soluble allofactor, an alloantigen shed by a graft, an antigen of an intracellular pathogen, an antigen of a viral vector used for gene therapy or gene vaccination, a tumor-associated antigen or an allergen.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B-, or T-, or NKT cell, and which is able, by said binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T lymphocyte. Whether an epitope is dominant, sub-dominant or minor depends on the immune reaction elicited against the epitope. Dominance depends on the frequency at which such epitopes are recognised by T cells and able to activate them, among all the possible T cell epitopes of a protein.

In an embodiment, the T cell epitope of an antigenic protein is an MHC class II T cell epitope or an NKT cell epitope.

The term “MHC class II T cell epitope” refers to a sequence, generally of +/−9 amino acids, which fits in the groove of the MHC II molecule. Within a peptide sequence representing a MHCII T cell epitope, the amino acids in the epitope are numbered P1 to P9, amino acids N-terminal of the epitope are numbered P-1, P-2 and so on, amino acids C terminal of the epitope are numbered P+1, P+2 and so on. Peptides recognised by MHC class II molecules and not by MHC class I molecules are referred to as MHC class II restricted T cell epitopes.

The term “NKT cell epitope” refers to a part of an antigenic protein that is specifically recognized and bound by a receptor at the cell surface of an NKT cell. In particular, a

NKT cell epitope is an epitope bound by CD1d molecules. The NKT cell epitope has a general motif [FWYHT]-X(2)-[VILM]-X(2)-[FWYHT]. Alternative versions of this general motif have at position 1 and/or position 7 the alternatives [FWYH], thus [FWYH]-X(2)-[VILM]-X(2)-[FWYH].

Alternative versions of this general motif have at position 1 and/or position 7 the alternatives [FWYT], [FWYT]-X(2)-[VILM]-X(2)-[FWYT]. Alternative versions of this general motif have at position 1 and/or position 7 the alternatives [FWY], [FWY]-X(2)-[VILM]-X(2)-[FWY].

Regardless of the amino acids at position 1 and/or 7, alternative versions of the general motif have at position 4 the alternatives [ILM], e.g. [FWYH]-X(2)-[ILM]-X(2)-[FWYH] or [FWYHT]-X(2)-[ILM]-X(2)-[FWYHT] or [FWY]-X(2)-[ILM]-X(2)-[FWY].

“Natural killer T” or “NKT” cells constitute a distinct subset of non-conventional T lymphocytes that recognize antigens presented by the non-classical MHC complex molecule CD1d. Two subsets of NKT cells are presently described. Type I NKT cells, also called invariant NKT cells (iNKT), are the most abundant. They are characterized by the presence of an alpha- beta T cell receptor (TCR) made of an invariant alpha chain, Valpha14 in the mouse and Valpha24 in humans. This alpha chain is associated to a variable though limited number of beta chains. Type 2 NKT cells have an alpha-beta TCR but with a polymorphic alpha chain. However, it is apparent that other subsets of NKT cells exist, the phenotype of which is still incompletely defined, but which share the characteristics of being activated by glycolipids presented in the context of the CD1d molecule.

NKT cells typically express a combination of natural killer (NK) cell receptor, including NKG2D and NK1.1. NKT cells are part of the innate immune system, which can be distinguished from the adaptive immune system by the fact that they do not require expansion before acquiring full effector capacity. Most of their mediators are preformed and do not require transcription. NKT cells have been shown to be major participants in the immune response against intracellular pathogens and tumor rejection. Their role in the control of autoimmune diseases and of transplantation rejection is also advocated.

The recognition unit, the CD1d molecule, has a structure closely resembling that of the MHC class I molecule, including the presence of beta-2 microglobulin. It is characterized by a deep cleft bordered by two alpha chains and containing highly hydrophobic residues, which accepts lipid chains. The cleft is open at both extremities, allowing it to accommodate longer chains. The canonical ligand for CD1d is the synthetic alpha galactosylceramide (alpha GalCer). However, many natural alternative ligands have been described, including glyco- and phospholipids, the natural lipid sulfatide found in myelin, microbial phosphoinositol mannoside and alpha-glucuronosylceramide. The present consensus in the art (Matsuda et al (2008), Curr. Opinion Immunol., 20 358-368; Godfrey et al (2010), Nature rev. Immunol 11, 197-206) is still that CD1d binds only ligands containing lipid chains, or in general a common structure made of a lipid tail which is buried into CD1d and a sugar residue head group that protrudes out of CD1d.

The identification and selection of a T-cell epitope from antigenic proteins is known to a person skilled in the art.

To identify an epitope suitable in the context of the present invention, isolated peptide sequences of an antigenic protein are tested by, for example, T cell biology techniques, to determine whether the peptide sequences binds or fits into the binding cleft of a MHC class II molecule or a CD1d molecule, and/or elicit a T cell response (i.e. a T cell or NKT cell response). Those peptide sequences found to elicit a T cell response are defined as having T cell stimulating activity.

Binding affinity experiments to MHC class II or CD1d molecules can be performed to determine whether an epitope suitable in the context of the present invention fits into the binding cleft of the MHC class II molecule or the CD1d molecule. For instance, soluble HLA class II molecules or CD1d molecules are obtained by lysis of cells homozygous for a given class II or CD1d molecule. The latter is purified by affinity chromatography. Soluble class II or CD1d molecules are incubated with a biotin-labelled reference peptide produced according to its strong binding affinity for that class II or CD1d molecule. Peptides to be assessed for class II or CD1d binding are then incubated at different concentrations and their capacity to displace the reference peptide from its class II or CD1d binding is calculated by addition of neutravidin.

Non-natural (or modified) T-cell epitopes can further optionally be tested on their binding affinity to MHC class II or CD1d molecules as described above. Human T cell stimulating activity can further be tested by culturing T cells obtained from e.g. an individual having T1D, with a peptide/epitope derived from the auto-antigen involved in T1D and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxyterminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol. 409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174, Guan et al. (2003) Appl. Bioinformatics 2, 63-66 (MHCPred) and Singh and Raghava (2001) Bioinformatics 17, 1236-1237

(Propred). More particularly, such algorithms allow the prediction within an antigenic protein of one or more octa- or nonapeptide sequences which will fit into the groove of an MHC II molecule and this for different HLA types.

A CD1d binding motif in a protein can be identified by scanning a sequence for the above sequence motifs, either by hand, either by using an algorithm such as ScanProsite De Castro E. et al. (2006) Nucleic Adds Res. 34(Web Server issue):W362-W365.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes:HLA-A, HLA-B, HLA-C, HLA-DPA1, HLA-DPB1, HLA-DQA1, HLAs DQB1, HLA-DRA, and HLA-DRB1. In humans, the MHC is divided into three regions:Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2).

Class I MHC molecules are expressed on virtually all nucleated cells.

Peptide fragments presented in the context of class I MHC molecules are recognised by CD8+ T lymphocytes (cytolytic T lymphocytes or CTLs). CD8+ T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N-and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-1, P-2 and so on, amino acids C-terminal of the epitope are numbered P+1, P+2 and so on.

In the peptides of the present invention comprising an oxidoreductase motif, the motif is located such that, when the epitope fits into the binding cleft of the MHCII molecule or the CD1d molecule, the motif remains outside of the MHCII or CD1d receptor binding groove. The oxidoreductase motif is placed either immediately adjacent to the epitope sequence within the peptide [in other words a linker sequence of zero amino acids between motif and epitope], or is separated from the T cell epitope by a linker comprising an amino acid sequence of 7 amino acids or less. More particularly, the linker comprises 1, 2, 3, 4, 5, 6, or 7 amino acids. Preferred embodiments are peptides with a 0, 1, 2, 3 or 4 amino acid linker between epitope sequence and oxidoreductase motif sequence. More preferably, the linker is 4 amino acids. Apart from a peptide linker, other organic compounds can be used as linker to link the parts of the peptide to each other (e.g. the oxidoreductase motif sequence to the T cell epitope sequence).

The peptides of the present invention can further comprise additional short amino acid sequences N or C-terminally of the sequence comprising the T cell epitope and the oxidoreductase motif. Such an amino acid sequence is generally referred to herein as a ‘flanking sequence’. A flanking sequence can be positioned between the epitope and an endosomal targeting sequence and/or between the oxidoreductase motif and an endosomal targeting sequence. In certain peptides, not comprising an endosomal targeting sequence, a short amino acid sequence may be present N and/or C terminally of the oxidoreductase motif and/or epitope sequence in the peptide. More particularly a flanking sequence is a sequence of between 1 and 7 amino acids, sus as a sequence of 1, 2, 3, 4, 5, 6 or 7 amino acids, most particularly a sequence of 2 amino acids.

The immunogenic peptides of the present invention can vary substantially in length.

The length of the T cell epitope comprised in the immunogenic peptide may vary from 7 to 30 amino acids, preferably from 7 to 25 amino acids, more preferably from 7 to 20 amino acids, e.g. 7, 8, 9, 10, 11, 12, 13, 14, 15, 16, 17, 18, 19 or 20 amino acids.

In a more particular embodiment, the T cell epitope consists of a sequence of 7, 8, or 9 amino acids. In a further particular embodiment, the T-cell epitope is an epitope, which is presented to T cells by MHC-class II molecules [MHC class II restricted T cell epitopes]. Typically T cell epitope sequence refers to the octapeptide or more specifically nonapeptide sequence which fits into the cleft of an MHC II protein.

In a further particular embodiment, the T-cell epitope is an epitope, which is presented by CD1d molecules [NKT cell epitopes]. Typically NKT cell epitope sequence refers to the 7 amino acid peptide sequence which binds to and is presented by the CD1d protein.

The length of the immunogenic peptides of the present invention can be of between 10 and 75 amino acids, preferably between 10 and 50 amino acids, more preferably between 10 and 40 amino acids, more preferably between 10 and 30 amino acids, and even more preferably between 10 and 25 amino acids.

In a specific embodiment, the length of the immunogenic peptides of the present invention can vary from 10 or 12 amino acids, i.e. consisting of an epitope of 7-9 amino acids, adjacent thereto an oxidoreductase motif of 3 amino acids, up to 20, 25, 30, 40, 50 or 75 amino acids.

In a preferred embodiment, the length of the immunogenic peptides of the present invention can vary from 15 or 17 amino acids, i.e. consisting of an epitope of 7-9 amino acids, a linker of 4 amino acids, adjacent thereto the oxidoreductase motif of 4 amino acids, up to 20, 25, 30, 40, 50, or 75 amino acids.

A peptide may also comprise an endosomal targeting sequence of e.g. 40 amino acids, a flanking sequence of about 2 amino acids, an oxidoreductase motif as described herein of 4 amino acids, a linker of 4 amino acids and a T cell epitope peptide of 9 amino acids.

The ‘epitope-oxidoreductase motif’ more particularly has a length of 10, 11, 12, 13, 14, 15, 16, 17, 18 or 19 amino acids. Such peptides can optionally be coupled to an endosomal targeting signal of which the size is less critical.

In an embodiment, the antigenic protein is an auto-antigen, a soluble allofactor, an alloantigen shed by a graft, an antigen of an intracellular pathogen, an antigen of a viral vector used for gene therapy or gene vaccination, a tumor-associated antigen or an allergen.

An “auto-antigen” as used herein refers to a human or animal protein present in the body, which elicits an immune response within the same human or animal body, thereby inducing an autoimmune disease. Autoimmune diseases are broadly classified into two categories, organ-specific and systemic diseases. The precise aetiology of systemic auto-immune diseases is not identified. In contrast, organ-specific auto-immune diseases are related to a specific immune response including B and T cells, which targets the organ and thereby induces and maintains a chronic state of local inflammation. Examples of organ-specific auto-immune diseases include type I. diabetes, myasthenia gravis, thyroiditis and multiple sclerosis. In each of these conditions, a single or a small number of auto-antigens have been identified, including insulin, the acetylcholine muscle receptor, thyroid peroxidase and major basic protein, respectively.

An “allofactor” as used herein refers to a protein, peptide or factor (i.e. any molecule) displaying polymorphism when compared between two individuals of the same species, and, more in general, any protein, peptide or factor that induces an (alloreactive) immune response in the subject receiving the allofactor. The soluble allofactor may be a protein applied in replacement therapy, or a coagulation or fibrinolytic factor, or a hormone, or a cytokine or a growth factor, or an antibody used for therapeutic purposes. A non-limiting list of possible allofactors includes factor VIII, factor IX, staphylokinase, growth hormone, insulin, cytokines and growth factors (such as interferon-alpha, interferon-gamma, GM-CSF and G-CSF), antibodies for the modulation of immune responses (including anti-IgE antibodies in allergic diseases, anti-CD3 and anti-CD4 antibodies in graft rejection and a variety of autoimmune diseases, anti-CD20 antibodies in non-Hodgkin lymphomas), and erythropoietin in renal insufficiency.

The term “alloantigen shed by a graft” or “allograft antigen” when used herein refers to an antigen derived from (shed from and/or present in) a cell or tissue which, when transferred from a donor to a recipient, can be recognized and bound by an antibody or B or T-cell receptor of the recipient. Alloantigens are typically products of polymorphic genes. An alloantigen is a protein or peptide which, when compared between donor and recipient (belonging to the same species), displays slight structural differences. The presence of such donor antigen in the body of a recipient can elicit an immune response in the recipient. Such alloreactive immune response is specific for the alloantigen. Examples of alloantigens are minor histocompatibility antigens, major histocompatibility antigens or tissue-specific antigens.

An “antigen of an intracellular pathogen” may be any antigen derived from bacteria, mycobacteria or parasites with an intracellular life cycle. Bacteria and mycobacteria include Mycobacterium tuberculosis, and other mycobacteria pathogenic for humans or animals such as Yersiniae, Brucellae, Chlamydiae, Mycoplasmae, Rickettsiae, Salmonellae and Shigellae. Parasites include Plasmodiums, Leishmanias, Trypanosomas, Toxoplasma gandii, Listeria sp., Histoplasma sp.

The term “viral vector used for gene therapy or gene vaccination” refers to adenovirus, adeno-associated virus, herpes virus or poxvirus or viral vectors derived from any thereof. Alternatively, the viral vector can be a retrovirus (such as gamma-retrovirus), a lentivirus or a viral vector derived from any thereof. Antigens from viral vector used for gene therapy or gene vaccination can be proteins present in the viral vector, such as capsid proteins, or fragments thereof.

The term “tumor-associated antigen” refers to any protein, peptide or antigen associated with (carried by, produced by, secreted by, etc) a tumor or tumor cell(s). Tumor -associated antigens may be (nearly) exclusively associated with a tumor or tumor cell(s) and not with healthy normal cells or may be overexpressed (e.g., 10 times, 100 times, 1000 times or more) in a tumor or tumor cell(s) compared to healthy normal cells. More particularly a tumor-associated antigen is an antigen capable of being presented (in processed form) by MHC determinants of the tumor cell. Hence, tumor associated antigens are likely to be associated only with tumor or tumor cells expressing MHC molecules. The tumor-associated antigen may be chosen from oncogenes, proto-oncogenes, viral proteins, surviving factors or clonotypic/idiotypic determinants. Such antigens are known and accepted in the art.

An “allergen” refers to a substance, usually a macromolecule or a proteic composition which elicits the production of IgE antibodies in predisposed, particularly genetically disposed, individuals (atopics) patients. Examples of allergens are pollen, stings, drugs, or food.

The term “food or pharmaceutical antigenic protein” refers to an antigenic protein present in a food or pharmaceutical product, such as in a vaccine.

In an embodiment, the immunogenic peptide according to the present invention is for use in medicine, preferably for use in treating and/or prevention of an autoimmune disease, of an infection with an intracellular pathogen, of a tumor, of an allograft rejection, or of an immune response to a soluble allofactor, to an allergen exposure or to a viral vector used for gene therapy or gene vaccination.

It has been shown that upon administration (i.e. injection) to a mammal of a peptide comprising an oxidoreductase motif and an MHC class II T-cell epitope (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells.

Additionally, it has been shown that upon administration (i.e. injection) to a mammal of a peptide comprising an oxidoreductase motif and an NKT-cell epitope (or a composition comprising such a peptide), the peptide elicits the activation of T cells recognising the antigen derived T cell epitope and provides an additional signal to the T cell through binding to the CD1d surface receptor. This activation results in NKT cells acquiring cytolytic properties for the cell presenting the T cell epitope.

In this way, the peptides or composition comprising the peptides described in the present invention, which contain an antigen-derived T cell epitope and, outside the epitope, an oxidoreductase motif can be used for direct immunisation of mammals, including human beings. The invention thus provides peptides of the invention or derivatives thereof, for use as a medicine. Accordingly, the present invention provides therapeutic methods which comprise administering one or more peptides according to the present invention to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with oxidoreductase properties, and further optionally also comprising a sequence to facilitate the uptake of the peptide into late endosomes for efficient MHC-class II presentation or CD1d receptor binding, elicit cytolytic CD4+ T-cells or NKT cells respectively.

The immunogenic properties of the peptides of the present invention are of particular interest in the treatment and prevention of immune reactions.

Peptides described herein are used as medicament, more particularly used for the manufacture of a medicament for the prevention or treatment of an immune disorder in a mammal, more in particular in a human.

The present invention describes methods of treatment or prevention of an immune disorder of a mammal in need for such treatment or prevention, by using the peptides of the invention, homologues or derivatives thereof, the methods comprising the step of administering to said mammal suffering or at risk of an immune disorder a therapeutically effective amount of the peptides of the invention, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases.

The peptides of the invention or a pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide according to the invention or a pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 μg or between 850 and 1300 μg, depending on the condition of the patient and severity of disease. Dosage regimen can comprise the administration in a single dose or in 2, 3, 4, 5, or more doses, either simultaneously or consecutively. Exemplary non-limiting administration schemes are the following:
A low dose scheme comprising the SC administration of 50 μg of peptide in two separate injections of 25 μg each (100 μL each) followed by three consecutive injections of 25 μg of peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of peptide as two separate injections of 37.5 μg each (150 μL each).
A high dose scheme comprising the SC administration of 450 μg of peptide in two separate injections of 225 μg each (900 μL each) followed by three consecutive administrations of 225 μg of peptide as two separate injections of 112.5 μg each (450 μL each).

An exemplary dose scheme of an immunogenic peptide comprising a known oxidoreductase motif and a T-cell epitope can be found on ClinicalTrials.gov under Identifier NCT03272269.

In a preferred embodiment, the oxidoreductase motif is located N-terminally from the epitope. Alternatively, the oxidoreductase motif may be located C-terminally from the epitope.

In a preferred embodiment, [C₁S₁T₁] or [C₂S₂T₂] in the oxidoreductase motif of the immunogenic peptide of the present invention corresponds to the N- or C-terminal end of the immunogenic peptide. That means in case the oxidoreductase motif is located N-terminally from the epitope, there is no other amino acid located N-terminally from [C₁S₁T₁]. In case the oxidoreductase motif is located C-terminally from the epitope, that means there is no other amino acid located C-terminally from [C₂S₂T₂].

In a preferred embodiment, the immunogenic peptide according to the present invention has a T-cell epitope which does not naturally comprise a [CST] residue within its sequence and/or within a region of 11 amino acids N-terminally or C-terminally of the T-cell epitope.

In another preferred embodiment, the immunogenic peptide according to present invention has an oxidoreductase motif which does not naturally occur within a region of 11 amino acids N-terminally or C-terminally of the T-cell epitope in said antigenic protein.

In a further preferred embodiment, the immunogenic peptide according to the present invention has a T-cell epitope which does not naturally comprise said oxidoreductase motif.

The term “natural” or “naturally” when referring to a peptide or epitope relates to the fact that the sequence is identical to a fragment of a naturally occurring protein (wild type or mutant) or fragment thereof. In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

In preferred embodiment, peptides according to the present invention are artificial peptides. Hence, the peptides of the present invention are preferably not natural (thus no fragments of proteins as such) but artificial peptides which contain, in addition to a T cell epitope, an oxidoreductase motif as described herein, whereby the oxidoreductase motif is immediately separated from the T cell epitope by a linker consisting of up to seven, most particularly up to four or up to two amino acids.

In this context, it is realised that peptide fragments are generated from antigens, typically in the context of epitope scanning. By coincidence such peptides fragments may naturally comprise in their sequence a T cell epitope (an MHC class II T cell epitope or an NKT cell epitope) with a [CST] residue within its sequence and/or within a region of at most 11 amino acids, at most 7 amino acids, at most 4 amino acids, at most 2 amino acids adjacent to said T cell epitope. In a preferred embodiment, such naturally occurring peptides are disclaimed. By coincidence such peptides fragments may also naturally comprise in their sequence a T cell epitope (an MHC class II T cell epitope or an NKT cell epitope) with an oxidoreductase motif as defined herein, preferably wherein C₁ is N-methylcysteine, within its sequence and/or within a region of at most 11 amino acids, at most 7 amino acids, at most 4 amino acids, at most 2 amino acids between said epitope and said oxidoreductase motif, or even 0 amino acids (in other words the epitope and oxidoreductase motif sequence are immediately adjacent to each other). In a preferred embodiment, such naturally occurring peptides are also disclaimed.

In a preferred embodiment, the one or two cysteines of the [C₁S₁T₁]-X_{n/m}-[C₂S₂T₂] motif are the only cysteines in the non-epitope part of the peptide. In a further preferred embodiment, the one or two cysteines of the [C₁S₁T₁]-X_{n/m}-[C₂S₂T₂] motif are the only cysteines of the immunogenic peptide.

In alternative embodiments, the T cell epitope may comprise any sequence of amino acids ensuring the binding of the epitope to the MHC cleft or to the CD1d molecule.

Where an epitope of interest of an antigenic protein comprises an oxidoreductase motif such as described herein within its epitope sequence, the immunogenic peptides according to the present invention comprise the sequence of an oxidoreductase motif as described herein and/or of another reducing sequence coupled N- or C-terminally to the epitope sequence such that (contrary to the oxidoreductase motif present within the epitope, which is buried within the cleft) the attached oxidoreductase motif can ensure the reducing activity.

The present invention also relates to a method for preparing an immunogenic peptide according to the invention, comprising the steps of:
a1) synthesizing said immunogenic peptide e.g. by conventional peptide synthesis for example using a conventional peptide synthesizer;
   or
a2) providing a peptide consisting of a T-cell epitope of an antigenic protein, and
b2) linking at the N- or C-terminal end of said peptide a compound of formula (III) or (IV) respectively, wherein R¹ to R⁷, m and n are as defined in claim 1 such that said compound of formula (III) or (IV) and said epitope are either adjacent to each other or separated by a linker of at most 7 amino acids;

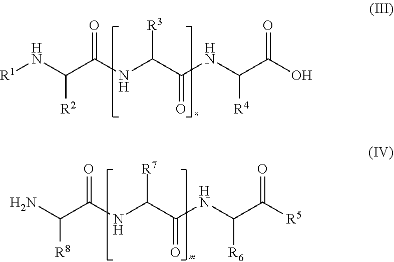

or
a3) providing a peptide consisting of a T-cell epitope of an antigenic protein, and
b3) linking at the N- or C-terminal end of said peptide with a compound of formula (V) or (VI) respectively, wherein R¹⁰ is hydrogen or R¹¹ is a NH₂ or OH and R² to R⁴ and R⁶ to R⁸, m and n are as defined in claim 1, such that said motif and said compound of formula (V) or (VI) are either adjacent to each other or separated by a linker of at most 7 amino acids, and replacing said R¹⁰ or R¹¹ of said compound of formula (V) or (VI) with at least one —CH₃—CH₂—C(═O)—, CH₃—C(═O)—, —CH₂—CH₃, or —CH₃ group,

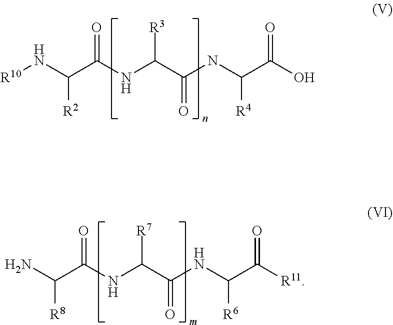

The peptides can be generated by chemical peptide synthesis, recombinant expression methods or in more exceptional cases, proteolytic or chemical fragmentation of proteins.

Preferably, the peptides of the present invention can be prepared by chemical peptide synthesis, wherein peptides are prepared by C- to N-terminus coupling of the different amino acids. Chemical synthesis is particularly suitable for the inclusion of unnatural modifications such as D-amino acids or modified amino acids, e.g. N-acetylcysteine, N-methylcysteine, N-ethylcysteine or N-propionylcysteine.

Peptide synthesis can be done using any standard technology such as through a standard peptide synthesizer using solid phase peptide synthesis (SPPS). Said technology is described in detail in e.g. Curr Protoc Protein Sci. 2012 Aug; CHAPTER: Unit-18.1; Introduction to Peptide Synthesis; Maciej Stawikowski and Gregg B. Fields.

Chemical peptide synthesis methods are well described. Peptides can also be ordered from companies such as LifeTein, Eurogentec and other.

Peptide chemical synthesis can for example be performed as either Solid Phase Peptide Synthesis (SPPS) or as solution phase peptide synthesis. The best known SPPS methods are Fmoc/^{t}Bu and Boc/Bzl methods.

In Fmoc/tBu SPPS the reactive groups on the side-chains of amino acids are protected with the following groups: Trt (trityl) for Cys, Glu, Asn, His; tBuO (tert-butoxy) for Asp, Ser, Thr and Tyr; Boc (tert-butyloxycarbony) for Lys and Trp; Pbf (2,2,4,6,7-Pentamethyldihydrobenzofuran-5-sulfonyl) for Arg. Briefly, a Fmoc-AA is coupled to the polymeric resin beads by using activation reagents via its C-terminus. After coupling, a Fmoc group of the coupled amino acid is removed (usually by piperidine, or similar), and the next Fmoc-AA is coupled. By iterating the coupling and deprotecting cycles, the peptide chain is elongated to yield the desired peptide sequence. The peptide is removed from the resin, and the side-chain groups are deprotected (other than Fmoc, which is removed after coupling the last amino acid) by using the TFA (trifluoroacetic acid). The process is well known and described in, e.g. Amide bond formation and peptide coupling, Tetrahedron, 2005, 61, 10827-10852; Advances in Fmoc solid-phase peptide synthesis, J Pept Sci. 2016, 22, 9-27, incorporated by reference herein.

In another embodiment, peptides can also be chemically modified after synthesis (e.g. adding/deleting functional groups) using techniques known in the art. N-acetylation,

N-methylation, N-ethylation or N-propionylation of cysteine, or C-terminal substitution by acetyl, methyl, ethyl or propionyl groups of the C-terminal amide or acid groups of cysteine in the oxidoreductase motif can hence be performed after peptide synthesis.

In case of C-terminal substitution of cysteine wherein the C-terminus is in the form of an acid, substitution of acid group by methyl or ethyl is done by esterification. C-terminal esterification of peptides can be performed e.g. by attaching the C-terminal amino acid moiety to a resin (or other solid phase) via its side chain while having orthogonal protection groups on its C- and N-termini. After completing the elongation of the peptide sequence by solid phase peptide synthesis (SPPS), the C-terminus can be deprotected, and the esterification reaction can be performed while the peptide still attached to the resin.

Substitution of acid group by acetyl or propionyl is done by creating an anhydride. Creation of C-terminal anhydride of peptides can be performed e.g. by attaching the C-terminal amino acid moiety to a resin (or other solid phase) via its side chain while having orthogonal protection groups on its C- and N-termini. After completing the elongation of the peptide sequence by SPPS, the C-terminus can be deprotected, and the anhydride reaction can be performed while the peptide still attached to the resin.

In case of C-terminal substitution of cysteine wherein the C-terminus is in the form of an amide,
C-terminal alkylation can be achieved by using Indole AM resins (Ethyl, or Methyl). After removing the peptide from the resin, the resulting peptide would be in the form of N-methyl or N-Ethyl substituted C-terminal amides.

Substitution of amide group by acetyl or propionyl is done by reacting 4-nitrophenyl acetate or 4-nitrophenyl propionate with the free C-terminus of the peptide, while it is still attached to the resin.

N-acetylation of cysteine can be performed e.g. by reacting the acetic anhydride (CH₃CO)₂O with the free N-terminus of the fully side chain protected peptide in basic conditions. The method is described e.g. in Solid-phase peptide synthesis: from standard procedures to the synthesis of difficult sequences, Nat. Protoc, 2007, 3247-3256, incorporated by reference herein.

N-methylation of cysteine can be performed e.g. by two-step reductive amination reaction:
1. Reacting the free N-terminus of the fully side chain protected peptide with formaldehyde (CH₂O) to yield an imine;
2. Reductive amination by e.g. sodium borohydride (NaBH₄).

A similar method is described in Robust Chemical Synthesis of Membrane Proteins through a General Method of Removable Backbone Modification, J. Am. Chem. Soc. 2016, 138, 3553-3561, incorporated by reference herein.

N-ethylation of cysteine can be performed e.g. by two-step reductive amination reaction:
1. Reacting the free N-terminus of the fully side chain protected peptide with acetaldehyde (CH₃CHO) to yield an imine;
2. Reductive amination by e.g. sodium borohydride (NaBH₄).

The similar method is described in Robust Chemical Synthesis of Membrane Proteins through a General Method of Removable Backbone Modification, J. Am. Chem. Soc. 2016, 138, 3553-3561, incorporated by reference herein.

N-propionylation of cysteine can be performed e.g. by reacting the propionic anhydride ((CH₃CH₂CO)₂O) with the free N-terminus of the fully side chain protected peptide in basic conditions. The similar method is described in Solid-phase peptide synthesis: from standard procedures to the synthesis of difficult sequences, Nat. Protoc, 2007, 3247-3256, incorporated by reference herein.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T or NKT cells which are cytolytic via an apoptotic pathway for antigen presenting cells presenting the antigen which contains the epitope sequence which is also present in the peptide with the oxidoreductase motif.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the oxidoreductase motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment.

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “allergic diseases” or “allergic disorders” as used herein refer to diseases characterised by hypersensitivity reactions of the immune system to specific substances called allergens (such as pollen, stings, drugs, or food). Allergy is the ensemble of signs and symptoms observed whenever an atopic individual patient encounters an allergen to which he has been sensitised, which may result in the development of various diseases, in particular respiratory diseases and symptoms such as bronchial asthma. Various types of classifications exist and mostly allergic disorders have different names depending upon where in the mammalian body it occurs. “Hypersensitivity” is an undesirable (damaging, discomfort-producing and sometimes fatal) reaction produced in an individual upon exposure to an antigen to which it has become sensitised; “immediate hypersensitivity” depends of the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases.

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal. Alternatively, where the administration is through gene-therapy, the amount of naked DNA or viral vectors is adjusted to ensure the local production of the relevant dosage of the peptide of the invention, derivative or homologue thereof.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X or B, is used for a position where any amino acid is accepted. Alternative amino acids can be indicated by listing the acceptable amino acids for a given position, between square brackets (‘[]’). For example: [CST] stands for one amino acid selected from Cys, Ser or Thr, i.e. [CST] encompasses, either one of cysteine, serine, or threonine. Amino acids which are excluded as alternatives can be indicated by listing them between curly brackets (‘{ }’). For example: {AM} stands for any amino acid except Ala and Met. The different elements in a motif are optionally separated from each other by a hyphen (-). To distinguish between the amino acids, those outside the oxidoreductase motif can be called external amino acids, those within the oxidoreductase motif are called internal amino acids.

A peptide, comprising a T cell epitope, e.g. an MHC class II T-cell epitope or an NKT-cell epitope (or CD1d binding peptide epitope) and a modified peptide motif sequence, having reducing activity is capable of generating a population of antigen-specific cytolytic CD4+ T-cells, or cytolytic NKT-cells towards antigen-presenting cells.

Accordingly, in its broadest sense, the invention relates to peptides which comprise at least one T-cell epitope (MHC class II T-cell epitope or an NKT-cell epitope) of an antigen (self or non-self) with a potential to trigger an immune reaction, and an oxidoreductase sequence motif with a modified cysteine. The T cell epitope and the oxidoreductase motif sequence may be immediately adjacent to each other in the peptide or optionally separated by one or more amino acids (so called linker sequence). Optionally the peptide additionally comprises an endosome targeting sequence and/or additional “flanking” sequences.

The peptides of the invention comprise a T-cell epitope of an antigen (self or non self) with a potential to trigger an immune reaction, and an oxidoreductase motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

As explained in detail further on, the peptides of the present invention can be made by chemical synthesis, which also allows the incorporation of non-natural amino acids.

In certain embodiments of the present invention, peptides are provided comprising one epitope sequence and an oxidoreductase motif sequence. In further particular embodiments, the oxidoreductase motif occurs several times (1, 2, 3, 4 or even more times) in the peptide, for example as repeats of the oxidoreductase motif which can be spaced from each other by one or more amino acids or as repeats which are immediately adjacent to each other. Alternatively, one or more oxidoreductase motifs are provided at both the N and the C terminus of the T cell epitope sequence.

Other variations envisaged for the peptides of the present invention include peptides which contain repeats of a T cell epitope sequence wherein each epitope sequence is preceded and/or followed by the oxidoreductase motif (e.g. repeats of “oxidoreductase motif-epitope” or repeats of “oxidoreductase motif-epitope-oxidoreductase motif”).

Herein the oxidoreductase motifs can all have the same sequence but this is not obligatory. It is noted that repetitive sequences of peptides which comprise an epitope which in itself comprises the oxidoreductase motif will also result in a sequence comprising both the ‘epitope’ and a ‘oxidoreductase motif’. In such peptides, the oxidoreductase motif within one epitope sequence functions as an oxidoreductase motif outside a second epitope sequence.

The T cell epitope of the peptides of the present invention can correspond either to a natural epitope sequence of a protein or can be a modified version thereof, provided the modified T cell epitope retains its ability to bind within the MHC cleft or to bind the CD1d receptor, similar to the natural T cell epitope sequence. The modified T cell epitope can have the same binding affinity for the MHC protein or the CD1d receptor as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC or CD1d complex compensates for the lowered affinity of the modified epitope for the MHC or CD1d molecule.

The sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and corresponds to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. lmmunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Alternatively, the present invention relates to the production of peptides containing hydrophobic residues that confer the capacity to bind to the CD1d molecule. Upon administration, such peptides are taken up by APC, directed to the late endosome where they are loaded onto CD1d and presented at the surface of the APC. Said hydrophobic peptides being characterized by a motif corresponding to the general sequence [FWYHT]-X(2)-[VILM]-X(2)-[FWYHT]. Alternative versions of this general motif have at position 1 and/or position 7 the alternatives [FWYH], thus [FWYH]-X(2)-[VILM]-X(2)-[FWYH], in which positions P1 and P7 are occupied by hydrophobic residues such as phenylalanine (F) or tryptophan (W). P7 is however permissive in the sense that it accepts alternative hydrophobic residues to phenylalanine or tryptophan, such as threonine (T) or histidine (H). The P4 position is occupied by an aliphatic residue such as isoleucine (I), leucine (L) or methionine (M). The present invention relates to peptides made of hydrophobic residues which naturally constitute a CD1d binding motif. In some embodiment, amino acid residues of said motif are modified, usually by substitution with residues which increase the capacity to bind to CD1d. In a specific embodiment, motifs are modified to fit more closely with the general motif [FW]-xx-[ILM]-xx-[FWTH]. More particularly, peptides are produced to contain a F or W at position 7.

Accordingly, the present invention envisages peptides of antigenic proteins and their use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides of the invention are generated by coupling a reducing compound, more particularly a reducing oxidoreductase motif as described herein, N-terminally or C-terminally to a T cell epitope of the antigenic protein (either directly adjacent thereto or with a linker of at most 10, more particularly at most 7 amino acids). Moreover the T cell epitope sequence of the protein and/or the oxidoreductase motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or ‘naturally occurring’.

The invention further relates to a method for obtaining a population of antigen-specific cytolytic CD4+ T cells, against APC presenting said antigen, the method comprising the steps of:
providing peripheral blood cells;
contacting said cells with an immunogenic peptide according to the invention
expanding said cells in the presence of IL-2.

The invention also relates to a method for obtaining a population of antigen-specific NKT cells, the method comprising the steps of:
providing peripheral blood cells;
contacting said cells with an immunogenic peptide according to the invention
expanding said cells in the presence of IL-2.

The invention also further relates to a method for obtaining a population of antigen-specific cytolytic CD4+ T cells, against APC presenting said antigen, the method comprising the steps of:
providing an immunogenic peptide according to the invention
administering said peptide to a subject; and
obtaining said population of antigen-specific cytolytic CD4+ T cells from said subject.

The invention also even further relates to a method for obtaining a population of antigen-specific NKT cells, the method comprising the steps of:
providing an immunogenic peptide according to the invention
administering said peptide to a subject; and
obtaining said population of antigen-specific NKT cells from said subject.

The present invention hence provides methods for generating antigen-specific cytolytic CD4+ T-cells (when using an immunogenic peptide as disclosed herein comprising an

MHC class II epitope), or antigen-specific cytolytic NKT-cells (when using an immunogenic peptide as disclosed herein comprising an NKT cell epitope binding the CD1d molecule) either in vivo or in vitro.

The mechanism of action of immunogenic peptides comprising a standard oxidoreductase motif and an MHC class II T-cell epitope is substantiated with experimental data disclosed in the above cited PCT application W02008/017517 and publications of the present inventors. The mechanism of action of immunogenic peptides comprising a standard oxidoreductase motif and a CD1d binding NKT-cell epitope is substantiated with experimental data disclosed in the above cited PCT application WO2012/069568 and publications of the present inventors.

Cytolytic CD4 +T cells as obtained in the present invention, induce APC apoptosis after MHC-class II dependent cognate activation, affecting both dendritic and B cells, as demonstrated in vitro and in vivo, and suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

The immunogenic peptides of the invention containing hydrophobic residues that confer the capacity to bind to the CD1d molecule. Upon administration, are taken up by APC, directed to the late endosome where they are loaded onto CD1d and presented at the surface of the APC. Once presented by CD1d molecule, the oxidoreductase motif in the peptides enhances the capacity to activate NKT cells, becoming cytolytic NKT cells. Said immunogenic peptides activate the production of cytokine, such as IFN-gamma, which will activate other effector cells including CD4+ T cells and nCD8+ T cells. Both CD4+ and CD8+ T cells can participate in the elimination of the cell presenting the antigen as discussed in detail in WO2012/069568.

The present invention describes in vivo methods for the production of the antigen-specific cytolytic CD4+ T cells or NKT cells. A particular embodiment relates to the method for producing or isolating the CD4+ T cells or NKT cells by immunising animals (including humans) with the peptides of the invention as described herein and then isolating the CD4+ T cells or NKT cells from the immunised animals.

The present invention also describes in vitro methods for the production of antigen specific cytolytic CD4+ T cells or NKT cells towards APC. The present invention provides methods for generating antigen specific cytolytic CD4+ T cells and NKT cells towards APC.

In one embodiment, methods are provided which comprise the isolation of peripheral blood cells, the stimulation of the cell population in vitro by an immunogenic peptide according to the invention and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4 + T cells or NKT cells, obtainable by the methods of the present invention, are of particular interest for use as a medicament, particularly for use in the treatment and/or prevention of an autoimmune disease, an infection with an intracellular pathogen, a tumor, an allograft rejection, or an immune response to a soluble allofactors, to an allergen exposure or to a viral vector used for gene therapy or gene vaccination.

Both the use of allogenic and autogeneic cells are envisaged.

In one embodiment, the invention provides ways to expand specific NKT cells, with as a consequence increased activity comprising, but not limited to:
(i) increased cytokine production
(ii) increased contact- and soluble factor-dependent elimination of antigen-presenting cells. The result is therefore a more efficient response towards intracellular pathogens, autoantigens, allofactors, allergens, tumor cells and more efficient suppression of immune responses against graft and viral proteins used in gene therapy/gene vaccination.

The present invention also relates to the identification of NKT cells with required properties in body fluids or organs. The method comprises identification of NKT cells by virtue of their surface phenotype, including expression of NK1.1, CD4, NKG2D and CD244. Cells are then contacted with NKT cell epitopes defined as peptides able to be presented by the CD1d molecule. Cells are then expanded in vitro in the presence of IL-2 or IL-15 or IL-7.

Isolated cytolytic CD4+ T cells or NKT cells or cell populations, more particularly antigen-specific cytolytic CD4+ T cell or NKT cell populations generated as described are also used for the manufacture of a medicament for the prevention or treatment of immune disorders. Methods of treatment by using the isolated or generated cytolytic CD4+ T cells or NKT cells are disclosed.

As explained in WO2008/017517 cytolytic CD4+ T cells towards APC can be distinguished from natural Treg cells based on expression characteristics of the cells. More particularly, a cytolytic CD4 + T cell population demonstrates one or more of the following characteristics compared to a natural Treg cell population:
an increased expression of surface markers including CD103, CTLA-4, Fasl and ICOS upon activation, intermediate expression of CD25, expression of CD4, ICOS, CTLA-4, GITR and low or no expression of CD127 (IL7-R), no expression of CD27, expression of transcription factor T-bet and egr-2 (Krox-20) but not of the transcription repressor Foxp3, a high production of IFN-gamma and no or only trace amounts of IL-10, IL-4, IL-5, IL-13 or TGF-beta.

Further the cytolytic T cells express CD45R0 and/or CD45RA, do not express CCR7, CD27 and present high levels of granzyme B and other granzymes as well as Fas ligand.

As explained in WO2008/017517 cytolytic NKT cells against towards APC can be distinguished from non-cytolytic NKT cells based on expression characteristics of the cells. More particularly, a cytolytic CD4+ NKT cell population demonstrates one or more of the following characteristics compared to a non-cytolytic NKT cell population: expression of NK1.I, CD4, NKG2D and CD244.

The peptides of the invention will, upon administration to a living animal, typically a human being, elicit specific T cells exerting a suppressive activity on bystander T cells.

In specific embodiments the cytolytic cell populations of the present invention are characterised by the expression of FasL and/or Interferon gamma. In specific embodiments the cytolytic cell populations of the present invention are further characterised by the expression of GranzymeB.

This mechanism also implies that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules or CD1d molecules in the vicinity of T cells activated by peptides of the invention.

The invention also relates to a method of treating and/or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of administering the immunogenic peptide according to the invention or the cell population of the invention to said individual.

The invention further relates to a method of treating or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of:
providing peripheral blood cells of said individual,
contacting said cells with an antigenic peptide according to the invention
expanding said cells, and
administering said expanded cells to said individual.

For medicinal use or methods of treatment or prevention purposes, the peptides can be part of a pharmaceutical composition. As an example described further herein of a pharmaceutical composition, a peptide according to the invention is adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class II or CD1d molecules and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers.

The present invention relates to pharmaceutical compositions, comprising, as an active ingredient, one or more peptides according to the invention, in a mixture with a pharmaceutically acceptable carrier. The pharmaceutical composition of the present invention should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The term “pharmaceutically acceptable carrier” as used herein means any material or substance with which the active ingredient is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders. Suitable pharmaceutical carriers for use in the pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one- step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water soluble soaps and water-soluble synthetic surface-active agents. Suitable soaps are alkaline or alkaline-earth metal salts, unsubstituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanylphosphatidylcholine, dipalmitoylphoshatidylcholine and their mixtures.

Suitable non-ionic surfactants include polyethoxylated and poly propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw”, 2 d ed. (Hanser Verlag, Vienna, 1981) and “Encyclopaedia of Surfactants, (Chemical Publishing Co., New York, 1981). Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polyniethyl methacrylate and the other above described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments.

In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

The peptides of the present invention can also be used in diagnostic in vitro methods for detecting class II restricted CD4+ T cells in a sample. In this method a sample is contacted with a complex of an MHC class II molecule and a peptide according to the present invention. The CD4+ T cells are detected by measuring the binding of the complex with cells in the sample, wherein the binding of the complex to a cell is indicative for the presence of CD4+ T cells in the sample. The complex can be a fusion protein of the peptide and an MHC class II molecule. Alternatively MHC molecules in the complex are tetramers. The complex can be provided as a soluble molecule or can be attached to a carrier.

The peptides of the present invention can also be used in diagnostic in vitro methods for detecting NKT cells in a sample. In this method a sample is contacted with a complex of a CD1d molecule and a peptide according to the present invention. The NKT cells are detected by measuring the binding of the complex with cells in the sample, wherein the binding of the complex to a cell is indicative for the presence of NKT cells in the sample. The complex can be a fusion protein of the peptide and a CD1d molecule.

The present invention will now be illustrated by means of the following examples which are provided without any limiting intention. Furthermore, all references described herein are explicitly included herein by reference.

### EXAMPLES

### Example 1

### Peptide Design

In order to assess the effect of the N-modification of N-terminal cysteine (i.e. when oxidoreductase motif is placed at the N-terminus of the peptide) or of the alkylation of the C-terminal amide group of C-terminal cysteine (i.e. when oxidoreductase motif is placed at the C-terminus of the peptide) on the activity of the oxidoreductase motif in connection to a T-cell epitope, the following peptides (tables 1, 2, 3, 4, 5,6, 7 and 8) were synthesised and compared to an immunogenic peptide comprising a non modified cysteine. Peptides displayed in table 1 comprise a C₁PYC oxidoreductase motif, wherein C₁ is N-modified or not and corresponds to the N-terminus of the peptide, linked to a MHC class II T cell epitope of insulin. Peptides displayed in table 2 comprise a C₁PYC oxidoreductase motif, wherein C₁ is N-modified or not and corresponds to the N-terminus of the peptide, linked to a MHC class II T cell epitope of the Tetanus toxin.

Peptides displayed in table 3 comprise a C₁HGC oxidoreductase motif, wherein C₁ is N-modified or not and corresponds to the N-terminus of the peptide, linked to a MHC class II T cell epitope of insulin. Peptides displayed in table 4 comprise a C₁PYC oxidoreductase motif, wherein C₁ is N-modified or not and corresponds to the N-terminus of the peptide, linked to a NKT cell epitope of hexon protein of adenovirus (Ad5). Peptides displayed in table 5 comprise a C₁PYC oxidoreductase motif, wherein

C₁ is N-modified or not and corresponds to the N-terminus of the peptide, linked to a MHC class II T cell epitope of the myelin oligodendrocyte (MOG) protein.

Peptides displayed in table 6 comprise a C₁HGC oxidoreductase motif, wherein C₁ is N-acetylated or not and corresponds to the N-terminus of the peptide, linked to a MHC class II T cell epitope of MOG.

Peptides displayed in table 7 comprise a CPYC₁ oxidoreductase motif, wherein C₁ is alkylated (ethylated or methylated) on the C-terminal amide group or not and corresponds to the C-terminus of the peptide, linked to a MHC class II T cell epitope of MOG. Peptides displayed in table 8 comprise a C₁GC oxidoreductase motif, wherein C₁ is N-modified or not and corresponds to the N-terminus of the peptide, linked to a MHC class II T cell epitope of insulin.

TABLE 1
Immunogenic peptides with N-modified cysteine based on the CPYC
motif placed at the N-terminus of the peptide and an insulin T cell
epitope. —NH₂ corresponds to C-terminal amid group.

| Peptide- | Oxidoreductase | | | |
|---|---|---|---|---|
| No° | Motif | Linker | T-Epitope | C-term |
| 1 | C-PYC | SLQP | LALEGSLQK | RG —NH₂ |
| 2 | N-acetyl C-PYC | SLQP | LALEGSLQK | RG —NH₂ |
| 3 | N-methyl C-PYC | SLQP | LALEGSLQK | RG —NH₂ |
| 4 | N-ethyl C-PYC | SLQP | LALEGSLQK | RG —NH₂ |
| 5 | N-propionyl C-PYC | SLQP | LALEGSLQK | RG —NH₂ |

TABLE 2
Immunogenic peptides with N-modified cysteine based on the CPYC
motif placed at the N-terminus of the peptide and a Tetanus toxin T cell
epitope. —NH₂ corresponds to C-terminal amid group.

| Peptide- | Oxidoreductase | | | |
|---|---|---|---|---|
| No° | Motif | Linker | T-Epitope | C-term |
| 6 | C-PYC | V | QYIKANSKFIGIT | EL —NH₂ |
| 7 | N-acetyl C-PYC | V | QYIKANSKFIGIT | EL —NH₂ |
| 8 | N-methyl C-PYC | V | QYIKANSKFIGIT | EL —NH₂ |
| 9 | N-ethyl C-PYC | V | QYIKANSKFIGIT | EL —NH₂ |
| 10 | N-propionyl C-PYC | V | QYIKANSKFIGIT | EL —NH₂ |

TABLE 3
Immunogenic peptides with N-modified cysteine based on the CHGC
motif placed at the N-terminus of the peptide and an insulin T cell
epitope. —NH₂ corresponds to C-terminal amid group.

| Peptide- | Oxidoreductase | | | |
|---|---|---|---|---|
| No° | Motif | Linker | T-Epitope | C-term |
| 11 | C-HGC | SLQP | LALEGSLQK | RG —NH₂ |
| 12 | N-acetyl C-HGC | SLQP | LALEGSLQK | RG —NH₂ |
| 13 | N-methyl C-HGC | SLQP | LALEGSLQK | RG —NH₂ |
| 14 | N-ethyl C-HGC | SLQP | LALEGSLQK | RG —NH₂ |
| 15 | N-propionyl C-HGC | SLQP | LALEGSLQK | RG —NH₂ |

TABLE 4
Immunogenic peptides with N-modified cysteine based
on the CPYC motif placed at the N-terminus of the peptide
and an hexon protein of adenovirus (Ad5) NKT cell
epitope. —NH₂ corresponds to C-terminal amid group.

| Peptide- | Oxidoreductase | | | |
|---|---|---|---|---|
| No° | Motif | Linker | T-Epitope | C-term |
| 16 | C-PYC | GG | FIGLMYY —NH₂ | |
| 17 | N-acetyl C-PYC | GG | FIGLMYY —NH₂ | |
| 18 | N-methyl C-PYC | GG | FIGLMYY —NH₂ | |
| 19 | N-ethyl C-PYC | GG | FIGLMYY —NH₂ | |
| 20 | N-propionyl C-PYC | GG | FIGLMYY —NH₂ | |

TABLE 5
Immunogenic peptides with N-modified cysteine based on the
CPYC motif placed at the N-terminus of the peptide and a MOG T cell
epitope. —NH₂ corresponds to C-terminal amid group.

| Peptide- | Oxidoreductase | | | |
|---|---|---|---|---|
| No° | Motif | Linker | T-Epitope | C-term |
| 21 | C-PYC | GW | YRSPFSRVV | HLYR —NH₂ |
| 22 | N-acetyl C-PYC | GW | YRSPFSRVV | HLYR —NH₂ |
| 23 | N-methyl C-PYC | GW | YRSPFSRVV | HLYR —NH₂ |
| 24 | N-ethyl C-PYC | GW | YRSPFSRVV | HLYR —NH₂ |
| 25 | N-propionyl C-PYC | GW | YRSPFSRVV | HLYR —NH₂ |

TABLE 6
Immunogenic peptides with N-modified cysteine based on the
CHGC motif placed at the N-terminus of the peptide and a MOG T cell
epitope. —NH₂ corresponds to C-terminal amid group.

| Peptide- | Oxidoreductase | | | |
|---|---|---|---|---|
| No° | Motif | Linker | T-Epitope | C-term |
| 26 | C-HGC | GW | YRSPFSRVV | HLYR —NH₂ |
| 27 | N-acetyl C-HGC | GW | YRSPFSRVV | HLYR —NH₂ |

TABLE 7
Immunogenic peptides with alkylated C-terminal amide
group of C-terminal cysteine of the CPYC motif placed at
the C-terminus of the peptide and a MOG T cell
epitope. —NH₂ and —NH— correspond to C-terminal amid group.

| Peptide- | | | | |
|---|---|---|---|---|
| No° | N-term | T-Epitope | Linker | Oxidoreductase Motif |
| 28 | GW | YRSPFSRVV | HLYR | CPY-C—NH₂ |
| 29 | GW | YRSPFSRVV | HLYR | CPY-C—NH-methyl |
| 30 | GW | YRSPFSRVV | HLYR | CPY-C—NH-ethyl |

TABLE 8
Immunogenic peptides with N-acetyl cysteine based on the
CGC motif placed at the N-terminus of the peptide and an insulin
epitope. —NH₂ corresponds to C-terminal amid group.

| Peptide- | Oxidoreductase | | | |
|---|---|---|---|---|
| No° | Motif | Linker | T-Epitope | C-term |
| 31 | C-GC | SLQP | LALEGSLQK | RG —NH₂ |
| 32 | N-acetyl C-GC | SLQP | LALEGSLQK | RG —NH₂ |
| 33 | N-methyl C-GC | SLQP | LALEGSLQK | RG —NH₂ |
| 34 | N-ethyl C-GC | SLQP | LALEGSLQK | RG —NH₂ |
| 35 | N-propionyl C-GC | SLQP | LALEGSLQK | RG —NH₂ |

### Example 2

### Assessment of the Reducing Activity of Peptides

The reductase activity of the peptides is determined using a fluorescent assay described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

Control experiments are performed with dithiotreitol (100% reducing activity) and water (0% reducing activity).

The peptides of the invention were tested for their reducing activity.

Control experiments are performed with DTT (dithiothreitol) which is assigned as 100 of reducing activity, and water (0% reducing activity).

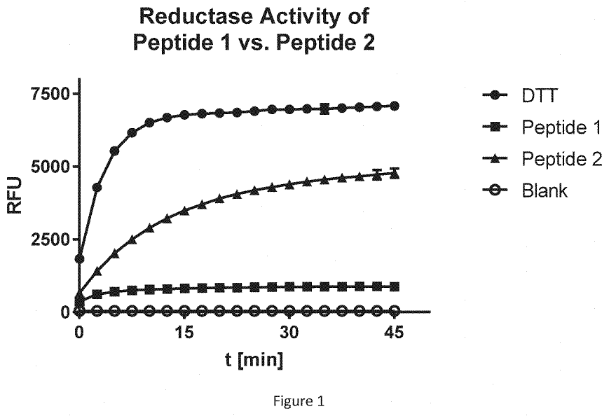

As displayed in FIG. 1, the peptide N-Acetyl-CPYCSLQPLALEGSLQKRG has a higher oxidoreductase activity than the control peptide CPYCSLQPLALEGSLQKRG without N-modified cysteine.

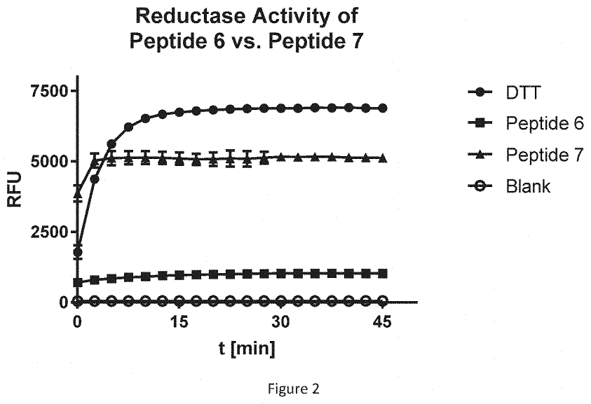

As displayed in FIG. 2, the peptide N-Acetyl-CPYCVQYIKANSKFIGITEL has a higher oxidoreductase activity than the control peptide CPYCVQYIKANSKFIGITEL without N-modified cysteine.

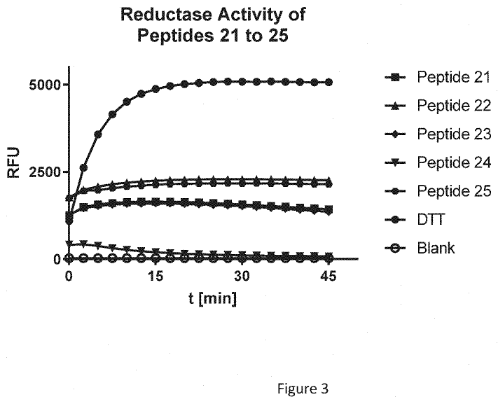

As displayed in FIG. 3, the peptides N-acetyl- or N-propionyl-CPYCGWYRSPFSRWHLYR have a higher oxidoreductase activity than the control peptide CPYCGWYRSPFSRWHLYR without N-modified cysteine. The peptide N-methyl-CPYCGWYRSPFSRWHLYR has an equivalent activity as compared to the control peptide without N-modified cysteine. The peptide N-ethyl-CPYCGWYRSPFSRWHLYR had no oxidoreductase activity.

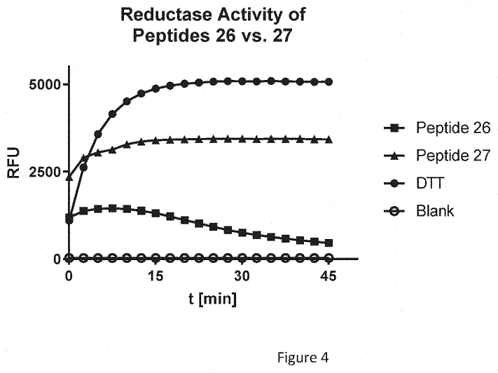

As displayed in FIG. 4, the peptides N-Acetyl—CHGCGWYRSPFSRWHLYR has a higher activity than the control peptide CHGCGWYRSPFSRWHLYR without N-modified cysteine.

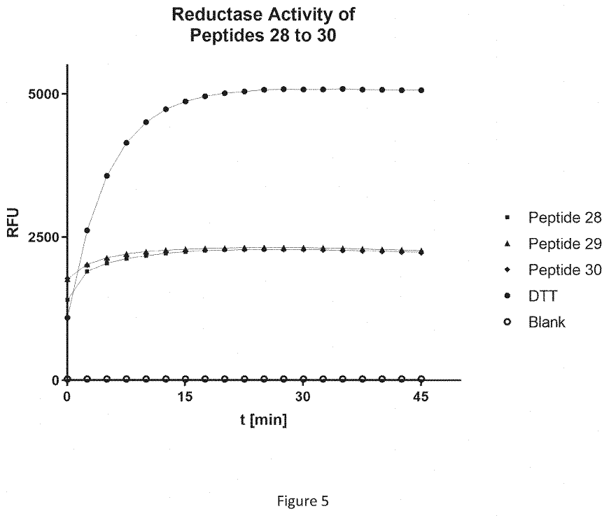

As displayed in FIG. 5, the peptides GWYRSPFSRVVHLYRCPYC-NH-methyl or —NH-ethyl have a slightly higher oxidoreductase activity than the control peptide GWYRSPFSRWHLYRCPYC-NH₂ without modified cysteine, especially at early time points.

### Example 3

### Assessment of the Capacity of Peptides Variants to Elicit Cytolytic CD4+ T Cells

The assess the capacity of peptides variants to elicit specific cytolytic CD4⁺ T cell, 2D2 transgenic mice with a TCR specific for myelin oligodendrocyte glycoprotein (MOG) were used. Three subcutaneous injections of peptides displayed in table 5 were performed at 12 days intervals on 2D2 mice with 50 μg of individual peptides variants adjuvanted with Alum. Fourteen days after the last injection, mice were sacrificed and splenocytes were prepared. In a first raw of experiment, splenic CD4+ T cells were stained for differentiation markers (CD44 and CD62L) to allow comparison of peptides potency to stimulate CD4+ T cells. The same cells were also stimulated with wild type peptide (devoided of thioredox motif) to allow detection of lytic molecules produced by these cells as granzymes A and B, FasL and the degranulation marker CD107a+b. It is expected that variants with a modified cysteine will be more potent at differentiating specific CD4+ T cells towards a cytolytic phenotype.

In another set of experiments, splenocytes (containing specific CD4+ T cells and antigen presenting cells, APC) were cultured in the presence or not of wild type peptide for 18 hours before staining for Annexin V expression and 7-MD together with antibodies recognizing CD19 and CD11c, as to allow detection of apoptosis and cell death in APC due to the expression of the CD4+ T cells with cytolytic potential following cognate interaction. It is expected that the highest proportion of APC cell death will be measured in splenocytes from mice injected with variants with a modified cysteine.

### Example 4

### Assessment of the Capacity of Peptides Variants to Reduce Disulphide Bridges at the Surface of CD4⁺ T Cells

Peptide variants were compared for their capacity to reduce disulphide bridges at the surface of specific CD4+ T cells.

Splenic CD4+ T cells were purified from 2D2 TCR transgenic animals and put in contact with different splenic APC preparations loaded with individual peptide variants displayed in table 5. After 30 minutes, cells were washed and stained with an anti-CD4 antibody together with a fluorescent maleimide reagent that reacts with reduced disulfides at the cell surface, before analysis by flow cytometry. It is expected that, when presented by APC, variants with a modified cysteine will be the most potent at targeting and reducing disulphide bridges at the surface of CD4+ T cells, as indicated by increased maleimide fluorescent signal intensity.

1. An immunogenic peptide, said immunogenic peptide comprising:
a) an oxidoreductase peptide motif of the general formula R¹—C¹—Xₙ—C²— (Formula Ib) or —C¹—Xₘ—C²—R⁵ (Formula IIb);
b) a T-cell epitope of an antigenic protein; and
c) a linker between a) and b) of between 0 and 7 amino acids;
wherein X corresponds to any amino acid moiety;
wherein n and m both are 2;
wherein the C-terminal hyphen (-) in formula (Ib) indicates the point of attachment to the amino group of the N-terminal end of the linker (c) or the epitope (b), and wherein the N-terminal hyphen (-) in formula IIb indicates the point of attachment to the carbonyl group of the C-terminal end of the linker (c) or the epitope (b);
wherein R¹ is selected from the group comprising CH₃—CH₂—C(═O)—, CH₃—C(═O)—, —CH₂—CH₃, and —CH₃;
wherein R⁵ is selected from the group comprising CH₃—CH₂—C(═O)—O—, CH₃—C(═O)—O—, —O—CH₂—CH₃, —O—CH₃, CH₃—CH₂—C(═O)—NH—, CH₃—C(═O)—NH—, —NH—CH₂—CH₃, and —NH—CH₃;
wherein R¹-C¹ represents a cysteine residue chemically modified through N-acetylation, N-methylation, N-ethylation or N-propionylation; and
wherein C²-R⁵ represent a cysteine residue chemically modified through C-terminal substitution by acetyl, methyl, ethyl or propionyl groups of it's C-terminal amide or acid groups.

2. The immunogenic peptide according to claim 1, wherein each X independently is selected from: H, R, and K.

3. The immunogenic peptide according to claim 1, wherein each X independently is selected from: Y or P.

4. The immunogenic peptide according to claim 1, wherein the oxidoreductase motif is of formula (Ib).

5. The immunogenic peptide according to claim 1, wherein said T cell epitope of an antigenic protein is an MHC class II T cell epitope or an NKT cell epitope, and/or wherein said epitope fits into the binding cleft of the MHC class II molecule or the CD1d molecule respectively.

6. The immunogenic peptide according to claim 1, having a length of between 10 and 75 amino acids, preferably between 10 and 50 amino acids, more preferably between 10 and 40 amino acids, more preferably between 10 and 30 amino acids, and even more preferably between 10 and 25 amino acids.

7. The immunogenic peptide according to claim 1, wherein the linker is of between 0 and 4 amino acids.

8. The immunogenic peptide according to claim 1, wherein said antigenic protein is an auto-antigen, a soluble allofactor, an alloantigen shed by a graft, an antigen of an intracellular pathogen, an antigen of a viral vector used for gene therapy or gene vaccination, a tumor-associated antigen or an allergen.

9. The immunogenic peptide according to claim 1, for use in medicine.

10. A method for treating and/or preventing an autoimmune disease, infection with an intracellular pathogen, tumor, allograft rejection, an immune response to a soluble allofactor, to an allergen exposure or to a viral vector used for gene therapy or gene vaccination comprising administering to a subject in need thereof an effective amount of the immunogenic peptide according to claim 1.

11. A method for preparing an immunogenic peptide according to claim 1, comprising the steps of: synthesizing said immunogenic peptide starting from natural amino acids and a chemically modified cysteine selected from the group consisting of: N-acetylated cysteine, N-methylated cysteine, N-ethylated cysteine, N-propionylated cysteine, or a cysteine in which its C-terminally C-terminal amide or acid groups have been substituted by acetyl, methyl, ethyl or propionyl groups.

12. A method for preparing an immunogenic peptide according to claim 1, comprising the steps of:
a2) providing a peptide consisting of a T-cell epitope (b) of an antigenic protein, optionally coupled to a linker (c) of between 0 and 7 amino acids.
b2) providing an oxidoreductase motif having the following general structure: C¹—Xₙ—C— or —C—Xₘ—C²,
wherein X corresponds to any amino acid moiety;
wherein n and m both are 2;
wherein the C-terminal hyphen (-) in formula (Ib) indicates the point of attachment to the amino group of the N-terminal end of the linker (c) or the epitope (b), and wherein the N-terminal hyphen (-) in formula IIb indicates the point of attachment to the carbonyl group of the C-terminal end of the linker (c) or the epitope (b); and
b3) chemically modifying said C¹ amino acid residue through N-acetylation, N-methylation, M-ethylation or N-propionylation, or
chemically modifying said C² amino acid residue through C-terminal substitution by acetyl, methyl, ethyl or propionyl groups of it's C-terminal amide or acid groups.

13. A method for obtaining a population of antigen-specific cytolytic CD4+ T cells, against APC presenting said antigen or a population of antigen-specific NKT cells, the method comprising the steps of:
providing peripheral blood cells;
contacting said cells with an immunogenic peptide according to claim 1, and
expanding said cells in the presence of IL-2.

14. A population of antigen-specific cytolytic CD4+ T cells against APC presenting said antigen or a population of antigen-specific NKT cells, obtained by the method of claim 13.

15. The population of antigen-specific cytolytic CD4+ T cells against APC presenting said antigen or a population of antigen-specific NKT cells according to claim 14, for use in medicine.

16. A method for treating and/or preventing an autoimmune disease, infection with an intracellular pathogen, tumor, allograft rejection, an immune response to a soluble allofactor, to an allergen exposure or to a viral vector used for gene therapy or gene vaccination comprising administering to a subject in need thereof an effective amount of the population of antigen-specific cytolytic CD4+ T cells against APC presenting said antigen or a population of antigen-specific NKT cells according to claim 14.
